(19) United States
(12) Patent Application Publication
(10) Pub. No.: US 2023/0033133 A1
(43) Pub. Date: Feb. 2, 2023

(54) VACCINE CONJUGATES

(71) Applicant: ULTIMOVACS AB, Uppsala (SE)

(72) Inventors: Sara MANGSBO, Uppsala (SE); Adrianus VALENTIJN, PG Oegstgeest (NL); Ferdinand OSSENDORP, CT Amstelveen (NL); Robert CORDFUNKE, CM Delft (NL); Jan DRIJFHOUT, AK Leiden (NL)

(21) Appl. No.: 17/781,826

(22) PCT Filed: Dec. 4, 2020
(86) PCT No.: PCT/EP2020/084765

§ 371 Date: Jun. 2, 2022

(30) Foreign Application Priority Data
Dec. 4, 2019 (GB) 1917699.9

(51) Int. Cl. A61K 39/00; A61K 47/64; A61P 35/00

(52) U.S. Cl. CPC A61K 39/001193; A61K 47/6415; A61K 47/646; A61P 35/00; A61K 2039/55544; A61K 2039/6037; A61K 2039/70; A61K 2039/884

(57) ABSTRACT

The present invention relates to conjugates comprising B- and T-cell epitopes, vaccine compositions comprising said conjugates, their use in the prevention and treatment of cancer, such as prostate cancer, as well as kits comprising the conjugates and/or vaccine compositions. Also claimed are particular T-cell epitope-containing antigenic peptides, and nucleic acids encoding them and constructs and vectors comprising such nucleic acids.

### FIELD OF THE INVENTION

The present invention relates to conjugates comprising B- and T-cell epitopes, vaccine compositions comprising said conjugates, their use in the prevention and treatment of cancer, such as prostate cancer, as well as kits comprising the conjugates and/or vaccine compositions. Also claimed are particular T-cell epitope-containing antigenic peptides, and nucleic acids encoding them and constructs and vectors comprising such nucleic acids.

### BACKGROUND OF THE INVENTION

Many effective vaccines in use today are vaccines against infections, in which the primary vaccine component is an attenuated or inactivated pathogen, for example the polio vaccine. Another effective vaccine strategy is the use of a toxoid, i.e. an inactivated form of a toxin, to stimulate an immune response against the toxin itself. Such vaccines are particularly useful against infections mediated by a single exotoxin virulence factor. Well-known and effective toxoid vaccines include the tetanus and diphtheria vaccines.

However, while such vaccines have often proved effective, these traditional approaches have been unsuccessful in the generation of vaccines against some important pathogens. Such approaches are also unsuitable for the generation of cancer vaccines, i.e. vaccines to prevent and/or treat cancer. Cancer vaccines are vaccinations intended to stimulate an immune response against cancer cells, using antigenic cancer markers. New approaches are required to generate cancer vaccines, and may also be helpful in the search for vaccines against infectious diseases. One such approach is peptide vaccines, in which a single peptide (generally less than 50 amino acids in length) which incorporates multiple T-cell epitopes is used as a vaccine antigen.

WO 2011/115483 discloses a vaccine conjugate comprising a peptide derived from tetanus toxin, conjugated to an antigen, immunogen or a vehicle comprising an antigen or immunogen.

The majority of people are vaccinated early in life against tetanus, particularly in the Western world. According to the World Health Organisation, in 2015 approximately 86% of all infants worldwide were vaccinated against tetanus, meaning that anti-tetanus antibodies are in circulation in a high proportion of the population. As described above, the tetanus vaccine uses tetanus toxoid (TTd), an inactivated form of the tetanus toxin (TTx). This means that the resultant circulating antibodies are specific for epitopes present in TTx/TTd.

TTx comprises a heavy chain (α-chain) and light chain (β-chain) connected by a disulphide bond. The N-terminal region of the TTx heavy chain (whose complete sequence is set forth in SEQ ID NO: 22) was previously found to contain important B- and T-cell epitopes (Raju et al. (1996), J. Autoimmun. 9:79-88; Fischer et al. (1994), Mol. Immunol. 31:1141-1148), including fragments comprising the sequence GITELKKL (SEQ ID NO: 23, corresponding to amino acids of 383-390 of the TTx heavy chain whose sequence is set forth in SEQ ID NO: 22), including the sequence FIGITELKKLESKINKVF (SEQ ID NO: 1).

Prostate cancer is the second most common type of cancer worldwide, and is the most common cancer to be diagnosed in men in over 80 countries, including the UK. Although prostate cancer is often slow-progressing and does not always require aggressive treatment, in 2012 it was nonetheless reported to be the cause of over 300,000 deaths worldwide. In particular for metastatic prostate cancer, treatment options are currently limited, and there is thus an unmet medical need for effective therapies. The present invention is directed to addressing this presently-unmet medical need.

Various prostate cancer antigens and epitopes thereof are known in the art and are disclosed for example in Younger et al. (2008), Prostate Cancer Prostatic Dis. 11: 334-341; McNeel et al. (2001), Cancer Res. 61: 5161-5167; Johnson & McNeel (2012), Prostate 72: 730-730; Matsueda et al. (2005), Clin. Cancer Res. 11: 6933-6943; Kiessling et al. (2012), Cancers 4: 193-217; Kiessling et al. (2008), Eur. Urol. 53: 694-708; Matera (2010) Cancer Treat. Rev. 36: 131-141; Qin et al. (2005), Immunol. Lett. 99: 85-93.

One antigenic protein associated with prostate cancer is glutamate carboxypeptidase 2 (GCPII). GCPII is a transmembrane protein over-expressed by prostate cancer cells (and which can also be expressed by other malignancies). As a serum-marker for prostate cancer it has limited use, due to its membrane-bound format, however imaging technologies have been developed with success and are PET-based. Vaccines exist that have shown partial clinical response in advanced disease using dendritic cells pulsed with GCPII peptides (Salgaller et al. (1998), Prostate 35(2): 144-151).

Another antigenic protein associated with prostate cancer is prostatic acid phosphatase (PAP). PAP is a both a cell-bound and a secreted glycoprotein which is produced by prostate cells and is primarily located in the prostate epithelium. In the progression to cancer, cell-bound PAP expression is decreased and soluble expression is enhanced, which can mark a transition to intermediate/high risk prostate cancer (Zimmerman (2009), Purinergic Signal. 5(3): 273-275).

### DISCLOSURE OF THE INVENTION

The present invention is directed to conjugates which comprise antigens comprising both CD8+ and CD4+ T-cell cancer epitopes, and to vaccine compositions based on such cancer epitopes.

In one aspect, the invention provides a conjugate comprising at least one B-cell epitope-containing peptide conjugated to a T-cell epitope-containing antigen, wherein:

(i) said at least one B-cell epitope-containing peptide comprises a minimal tetanus toxoid epitope (MTTE), said MTTE comprising:
(a) an amino acid sequence of at least 10 amino acids which are contiguous in SEQ ID NO: 22 and comprise the amino acid sequence GITELKKL set forth in SEQ ID NO: 23; or
(b) an amino acid sequence with at least 70% sequence identity to an amino acid sequence of (a);

wherein said B-cell epitope-containing peptide is not the complete tetanus toxin beta chain;

(ii) said T-cell epitope-containing antigen is a polypeptide comprising from N-terminus to C-terminus:
(a) a translocation peptide;
(b) a CD8+ T-cell cancer epitope; and
(c) a CD4+ T-cell cancer epitope;
   wherein a proteasome cleavage site may optionally be present between said CD8+ T-cell epitope and said CD4+ T-cell epitope; and

(iii) the N-terminus of said T-cell epitope-containing antigen is conjugated to said B-cell epitope-containing peptide; and wherein

(iv) the conjugation of the at least one B-cell epitope-containing peptide to the T-cell epitope-containing antigen is direct or indirect.

In one embodiment of the invention, the B-cell epitope-containing peptide is directly linked to the T-cell epitope-containing antigen.

In one embodiment of the invention, the T-cell epitope-containing antigen consists of, from N-terminus to C-terminus:

i) a translocation peptide;

ii) a CD8+ T-cell cancer epitope;

iii) a spacer; and

iv) a CD4+ T-cell cancer epitope;

wherein the spacer provides a proteasome cleavage site.

In an embodiment of the invention, the B-cell epitope-containing peptide is linked to the T-cell epitope-containing antigen via a linker.

In one embodiment of the invention, the linker is a peptide sequence or any other chemical group or moiety.

In one embodiment of the invention, the translocation peptide referred to in (ii)(a) above mediates TAP-driven transport of said T-cell epitope-containing antigen or said CD8+ T-cell epitope into the endoplasmic reticulum of a host cell.

In another aspect the invention provides a conjugate comprising at least one B-cell epitope-containing peptide conjugated to a T-cell epitope-containing antigen, wherein:

(i) said at least one B-cell epitope-containing peptide comprises a minimal tetanus toxoid epitope (MTTE), said MTTE comprising:
(a) an amino acid sequence of at least 10 amino acids which are contiguous in SEQ ID NO: 22 and comprise the amino acid sequence GITELKKL set forth in SEQ ID NO: 23; or
(b) an amino acid sequence with at least 70% sequence identity to an amino acid sequence of (a);

wherein said B-cell epitope-containing peptide is not the complete tetanus toxin beta chain;

(ii) said T-cell epitope-containing antigen comprises a CD8+ T cell cancer epitope and a CD4+ T cell cancer epitope, wherein the CD8+ T cell cancer epitope is selected from any one of SEQ ID NOs: 2-6, or an amino acid sequence with at least 65% sequence identity thereto; and the CD4+ T cell cancer epitope is selected from any one of SEQ ID NOs: 7-11, or an amino acid sequence with at least 75% sequence identity thereto; and

(iii) the N-terminus of said T-cell epitope-containing antigen is conjugated to said B-cell epitope-containing peptide.

In another aspect, the invention provides a conjugate comprising at least one B-cell epitope-containing peptide conjugated to a T-cell epitope-containing antigen, wherein:

(i) said at least one B-cell epitope-containing peptide comprises a minimal tetanus toxoid epitope (MTTE), said MTTE comprising:
(a) an amino acid sequence of at least 10 amino acids which are contiguous in SEQ ID NO: 22 and comprise the amino acid sequence GITELKKL set forth in SEQ ID NO: 23; or
(b) an amino acid sequence with at least 70% sequence identity to an amino acid sequence of (a);

wherein said B-cell epitope-containing peptide is not the complete tetanus toxin beta chain;

(ii) said T-cell epitope-containing antigen is a peptide comprising a 20-35 amino acid fragment of SEQ ID NO: 18, or an amino acid sequence with at least 70% sequence identity to such a fragment; and

(iii) the N-terminus of said T-cell epitope-containing antigen is conjugated to said B-cell epitope-containing peptide.

Yet another aspect of the invention is a vaccine composition comprising at least one conjugate of the invention.

In another aspect, the invention provides a conjugate or vaccine composition of the invention for use in therapy.

In another aspect, the invention provides a conjugate or vaccine composition of the invention for use in the treatment or prevention of cancer, such as prostate cancer.

One aspect of the invention is a method for the prevention or treatment of cancer, such as prostate cancer, comprising administering to a subject in need of such prevention or treatment a therapeutically-effective amount of a conjugate or vaccine composition as disclosed and claimed herein.

Yet another aspect of the invention is the use of a conjugate or vaccine composition as disclosed and claimed herein in the manufacture of a medicament for use in the prevention or treatment of cancer, such as prostate cancer.

Another aspect of the invention is a polypeptide comprising or consisting of an amino acid sequence set forth in any one of SEQ ID NOs: 13-17, or an amino acid sequence with at least 70% sequence identity thereto, wherein said polypeptide comprises from N-terminus to C-terminus:
(a) a translocation peptide;
(b) a CD8+ T-cell cancer epitope; and
(c) a CD4+ T-cell cancer epitope;

wherein an optional proteasome cleavage site can be present between said CD8+ T-cell cancer epitope and said CD4+ T-cell cancer epitope.

In one aspect of the invention, the translocation peptide may mediate TAP-driven transport of the polypeptide or at least of said CD8+ T-cell epitope into the endoplasmic reticulum of a host cell.

Another aspect of the invention is a polypeptide comprising or consisting of an amino acid sequence set forth in any one of SEQ ID NOs: 106-110, or an amino acid sequence with at least 70% sequence identity thereto, wherein said polypeptide comprises from N-terminus to C-terminus:
(a) a CD8+ T-cell cancer epitope; and
(b) a CD4+ T-cell cancer epitope;

wherein an optional proteasome cleavage site can be present between said CD8+ T-cell cancer epitope and said CD4+ T-cell cancer epitope.

The invention also provides a nucleic acid molecule comprising or consisting of a nucleotide sequence encoding a polypeptide of the invention, a construct comprising a nucleic acid molecule of the invention and a vector comprising a nucleic acid molecule or a construct of the invention.

In another aspect, the invention provides a kit, or a combination therapy product, comprising a vaccine composition of the invention and a second therapeutically active agent.

One aspect of the invention provides a method of producing a conjugate as herein disclosed and claimed, comprising the steps of:

(i) attaching at least one B-cell epitope-containing peptide to a core compound; and

(ii) attaching a T-cell epitope-containing antigen to the product of (i).

In an embodiment, the method comprises the steps of:

(i) attaching at least one B-cell epitope-containing peptide comprising a thiol group to a core compound comprising at least one maleimide group and a second functional group, wherein the thiol group of the B-cell epitope-containing peptide reacts with the maleimide group of the core compound to form an adduct in which the B-cell epitope-containing peptide is attached to the core compound via a succinimide group;

(ii) attaching a T-cell epitope-containing antigen comprising a reactive group capable of reacting to form a linkage with the second functional group of the core compound to the adduct of (i), by reacting the reactive group of the antigen with the second functional group of the core compound to form a conjugate comprising at least one B-cell epitope-containing peptide and a T-cell epitope-containing antigen each linked to the core compound;

(iii) opening the at least one succinimide ring of the core compound, wherein said ring-opening may occur before or after step (ii).

In an embodiment, the C-terminal amino acid of each B-cell epitope-containing peptide comprises a thiol group. In another embodiment, the N-terminal amino acid of each B-cell epitope-containing peptide comprises a thiol group. In another embodiment, the thiol group is provided on a molecule which is conjugated to the B-cell epitope-containing peptide, preferably to its N-terminal or C-terminal amino acid.

In an embodiment, the reactive group of the T-cell epitope-containing antigen is an azido group, which may be coupled to the T-cell epitope containing antigen via the N-terminal amino acid of said antigen, or via the C-terminal amino acid of said antigen.

The functional group in the core compound, which functional group is capable of reacting with the reactive group of the T-cell epitope-containing antigen, e.g. with an azido group, may be a group comprising an alkyne moiety, for example a cycloalkyne group, e.g. a C5-C10 cycloalkyne group such as a cyclooctyne group, e.g. a diphenyl cyclooctyne group.

In one embodiment, the thiol group of the B-cell epitope-containing peptide is provided by a C-terminal cysteine residue. In another embodiment, the ring-opening of step (iii) is by hydrolysis.

In yet another embodiment the core compound comprises one, two or at least three (e.g. 3) maleimide groups, and one, two or at least three (e.g. 3) B-cell epitope-containing peptides are attached thereto. The B-cell epitope-containing peptides may be the same or different.

A “B-cell epitope-containing peptide” as used herein is an antigen comprising an epitope recognised by an antibody.

The term “antigen” generally means any substance (most commonly a protein) which is able to induce an adaptive immune response, either humoral (antibody) or cellular. However, in the present disclosure, to distinguish the antigen of the conjugate comprising the B-cell epitope from the antigen of the conjugate comprising T-cell epitopes, the antigen comprising the B-cell epitope will be referred to throughout as a “B-cell epitope-containing peptide”, and the antigen comprising the T-cell epitope will be referred to throughout as a “T-cell epitope-containing antigen”.

As used herein, the term “peptide” is interchangeable with the term “polypeptide” and refers to a polymer of amino acids covalently linked by peptide bonds. A “peptide” or “polypeptide” may also include one or more modified amino acids, e.g. amino acids modified by myristylation, sulfation, glycosylation or phosphorylation.

The term “epitope” means a single immunogenic site within a given antigen that is sufficient to elicit an immune response in a subject, i.e. an epitope is an antigenic determinant, the (or a) specific section of an antigen actually bound by an antibody or B/T-cell receptor. Epitopes can be linear sequences or conformational epitopes (conserved binding regions) depending on the type of immune response. A T-cell epitope is thus a site in an antigen bound by a T-cell receptor, and a B-cell epitope is a site in an antigen bound by a B-cell receptor (or antibody).

In an embodiment, and as described in more detail below, a (separate) vaccine may be administered to the subject to induce an immune response against tetanus toxin (more specifically to induce the generation of antibodies by the subject to tetanus toxin) prior to the administration of the conjugate. Such a vaccine may contain, for example, tetanus toxoid, or a component of fragment of tetanus toxin, e.g. of the tetanus toxin heavy chain. Alternatively, anti-tetanus toxin (e.g. anti-TTd) antibodies may be passively administered, for example using an isolated IgG fraction from high titre anti-TTd donors.

In one aspect of the invention, a B-cell epitope-containing peptide used in a conjugate of the invention comprises a B-cell epitope from the TTx sequence, known as a Minimal Tetanus Toxin Epitope (MTTE).

In one embodiment of the invention, the MTTE present in a conjugate of the invention comprises or consists of:

(a) an amino acid sequence of at least 10 amino acids which are contiguous in SEQ ID NO: 22 and comprise the amino acid sequence GITELKKL set forth in SEQ ID NO: 23; or

(b) an amino acid sequence with at least 70, 75, 80, 85, 90, 95, 96, 97, 98 or 99% sequence identity to an amino acid sequence of (a).

SEQ ID NO: 22 corresponds to the TTx heavy chain. SEQ ID NO: 23 corresponds to amino acids 383-390 of the TTx heavy chain, i.e. amino acids 383-390 of SEQ ID NO: 22.

The MTTE may comprise or consist of at least 12 or at least 15 amino acids which are contiguous in SEQ ID NO: 22, such as at least 18 amino acids which are contiguous in SEQ ID NO: 22, and comprise the amino acid sequence GITELKKL set forth in SEQ ID NO: 23. The MTTE may comprise or consist of at most 20, 25, 30, 35, 40, 45 or 50 amino acids which are contiguous in SEQ ID NO: 22 and comprise the amino acid sequence GITELKKL set forth in SEQ ID NO: 23. In further embodiments of the invention, the MTTE may comprise or consist of an amino acid sequence which has at least 70, 75, 80, 85, 90, 95, 96, 97, 98 or 99% sequence identity to a sequence of at least 12, 15 or 18 amino acids which are contiguous in SEQ ID NO: 22 and comprise the amino acid sequence set forth in SEQ ID NO: 23. In still further embodiments of the invention, the MTTE may comprise or consist of an amino acid sequence which has at least 70, 75, 80, 85, 90, 95, 96, 97, 98 or 99% sequence identity to a sequence of at most 20, 25, 30, 35, 40, 45 or 50 amino acids which are contiguous in SEQ ID NO: 22 and comprise the amino acid sequence set forth in SEQ ID NO: 23.

In accordance with the present invention, an MTTE which comprises or consists of an amino acid sequence which has at least 70, 75, 80, 85, 90, 95, 96, 97, 98 or 99% sequence identity to a sequence of at least 10 amino acids which are contiguous in SEQ ID NO: 22 and comprises the amino acid sequence set forth in SEQ ID NO: 23 is referred to as a “variant” of a sequence fragment of SEQ ID NO: 22 (by sequence fragment of SEQ ID NO: 22 is meant a sequence of 10 or more amino acids which are contiguous in SEQ ID NO: 22 and comprise the sequence set forth in SEQ ID NO: 23 but which do not constitute the complete TTx heavy chain). When a B-cell epitope-containing peptide present in a conjugate of the invention comprises or consists of a variant of a sequence fragment of SEQ ID NO: 22, it is important that the variant sequence is recognised by anti-TTx antibodies. Whether a particular sequence is recognised by anti-TTx antibodies may be determined by any method known in the art. In an exemplary embodiment of the invention, a Tettox ELISA is used to determine anti-TTx antibody binding to an amino acid sequence. A “Tettox ELISA” as defined herein is an ELISA assay specific for anti-TTx antibodies.

A person skilled in the art will understand how to perform an ELISA assay to identify whether anti-TTx antibodies bind a particular variant sequence fragment of SEQ ID NO: 22 of interest. Such a sequence fragment may be generated by any method known in the art, e.g. chemical synthesis. Anti-TTx antibodies may be obtained as a polyclonal antibody serum from a human donor who has received the tetanus toxoid vaccine. An exemplary Tettox ELISA protocol is described in detail in WO 2011/115483, and as disclosed therein a Tettox ELISA may be performed as follows:

A 96-well plate (e.g. from Euro-Diagnostica, Arnhem, Netherlands) is coated with streptavidin and then blocked with PBS containing 5% BSA (200 μl/well, 1 hr, room temperature). The plate is then washed three times with PBS containing 0.05% polysorbate 20 (e.g. Tween® 20).

The plate is then coated with a biotinylated peptide-of-interest (i.e. a peptide comprising a variant sequence fragment of SEQ ID NO: 22), by incubation of the plate for 1 hr at room temperature with 100 μl/well of a 2 μg/ml solution of the biotinylated peptide in PBS containing 1% BSA. The plate is then washed three times with PBS containing 0.05% polysorbate 20, and the primary antibody applied. The primary antibody is applied by incubation of the plate for 1 hr at room temperature with 100 μl/well serum solution from a human subject as defined above (i.e. a subject who has received the tetanus toxoid vaccine). The serum may be diluted with PBS containing 1% BSA, e.g. the serum may be diluted at least 1:10, 1:50, 1:100, 1:200, 1:400, 1:500, 1:1000, 1:2000, 1:4000 up to 1:100,000 or more to determine the titre. The plate is then washed three times with PBS containing 0.05% polysorbate 20.

The secondary antibody is then applied, by incubation of each well for 1 hr at room temperature with an appropriate anti-human IgG antibody. The anti-human IgG antibody may be conjugated to horseradish peroxidase (HRP), e.g. mouse anti-human IgG-HRP monoclonal, clone G18-145, Becton Dickinson no. 555788 may be used. 100 μl/well of the secondary antibody solution is applied, at an appropriate dilution in PBS containing 0.05% polysorbate 20. The secondary antibody may be diluted in accordance with the manufacturer's instructions, e.g. by a factor of 1:1000, 1:2000, 1:5000, etc. The plate is then washed three times with PBS containing 0.05% polysorbate 20.

Antibody binding to the peptide of interest may be identified using any appropriate method known in the art, e.g. using ABTS (2,2′-azino-di-(3-ethylbenzthiazoline sulfonic acid)) with H₂O₂. Using ABTS, peroxidase activity is measured according to the optical density of the solution in each well at 415 nm, which may be measured using a microplate reader (e.g. a BIO-RAD Model 680).

A negative control, such as serum from a human subject without detectable anti-TTx antibodies, may be included in each plate. A solution of BSA may also be useful as a negative control. Yet another example of a suitable negative control is the primary antibody serum of interest used with a peptide-of-interest which has a scrambled MTTE sequence rather than a variant sequence fragment of SEQ ID NO: 22. An exemplary scrambled MTTE sequence has the amino acid sequence set forth in SEQ ID NO: 98, which corresponds to a scrambled version of SEQ ID NO: 1. A positive control may also be included. Such a positive control may be the native (wild-type) sequence of the variant sequence of interest. Both control and experimental assays may be performed in at least duplicate or triplicate.

The peptide-of-interest may individually be subjected to serum samples from at least 10, 12, 15, 20, 25, 30, 40, 50, 60, 70, 80, 100, 120, 150, 200 or 250 or more human subjects. The human subjects may be randomly selected, or may be human subjects that have a high titre of anti-TTx antibodies, e.g. at least 100 International Units (IU) per ml as determined using the Tettox ELISA as described above using a wild-type fragment of TTx as the peptide-of-interest.

An MTTE as described herein which comprises or consists of a variant of a sequence fragment of SEQ ID NO: 22 is bound by antibodies in at least 40, 45, 50, 55, 60, 65, 70, 75, 80, 85, 90, 95, 96, 97, 98 or 99% of the tested human serum samples. Alternatively, the MTTE is bound by antibodies in all of the tested human serum samples (i.e. 100% of the samples). The skilled person will understand how to determine whether an ELISA gives a positive result, indicating binding of the primary antibody to a peptide of interest. A peptide may be considered to be bound by antibodies in a serum sample if the determined optical density for that particular serum sample is at least 2.0, 2.5, 3.0, 3.5 or more times higher than the optical density determined for the negative control.

In the Tettox ELISA described above, the primary, anti-TTx antibody can be provided as purified anti-TTx antibody prepared from donors instead of directly in serum from a human subject. For instance TetaQuin® (Sanquin, Amsterdam, Netherlands) may be used. In this case, TetaQuin is diluted in varying concentrations in PBS containing 1% BSA, and 100 μl/well of the diluted TetaQuin is applied to the peptide-of-interest. The remainder of the procedure may be performed as detailed above.

In one embodiment, the MTTE present in a conjugate of the invention comprises or consists of the amino acid sequence set forth in SEQ ID NO: 1, or an amino acid sequence with at least 70, 75, 80, 85, 90, 95, 96, 97, 98 or 99% sequence identity to the sequence set forth in SEQ ID NO: 1. SEQ ID NO: 1 is an 18 amino acid sequence which corresponds to amino acids 381-398 of the TTx heavy chain (i.e. amino acids 381-390 of SEQ ID NO: 22). The sequence of SEQ ID NO: 23 is located at positions 3-10 of SEQ ID NO: 1. As detailed in WO 2011/115483, positions 3-5 and 11 of SEQ ID NO: 1 are of particular importance for its function in stimulating an immune response.

In other embodiments of the invention, the MTTE comprises or consists of an amino acid sequence with at least 70, 75, 80, 85, 90, 95, 96, 97, 98 or 99% sequence identity to the sequence set forth in SEQ ID NO: 1, in which the amino acids at positions corresponding to positions 3-5 and 11 of SEQ ID NO: 1 are unchanged from the amino acids at positions 3-5 and 11 of SEQ ID NO: 1.

In yet other embodiments the MTTE present in a conjugate of the invention comprises or consists of an amino acid sequence set forth in any one of SEQ ID NOs: 30-86, or an amino acid sequence with at least 70, 75, 80, 85, 90, 95, 96, 97, 98 or 99% sequence identity to any one of SEQ ID NOs: 30-86.

The B-cell epitope (MTTE)-containing peptide may comprise a spacer sequence N-terminal and/or C-terminal to the MTTE. An N-terminal spacer sequence may be used to separate the MTTE from the N-terminus of the B-cell epitope-containing peptide, while a C-terminal spacer may be used to separate the MTTE from the C-terminus of the B-cell epitope-containing peptide. Any part of the B-cell epitope-containing peptide which does not constitute part of the MTTE may be considered to be a spacer. In one embodiment of the invention, a spacer sequence is located C-terminal to the MTTE.

The spacer sequence may be of any length. In one embodiment of the invention, the spacer sequence is at least 5 amino acids in length and at most 20 amino acids in length, such as from 5 to 18 amino acids, or from 5 to 15, or from 5 to 12, or from 6 to 18, or from 6 to 15, or from 6 to 12, or from 8 to 18, or from 8 to 15, or from 8 to 12 amino acids. The spacer sequence may be any amino acid sequence.

In one embodiment of the invention, the spacer is at least 5 amino acids long and is not derived from the TTx sequence. The TTx protein is encoded as a single protein, in the form of a pro-toxin, which is subsequently cleaved to yield the heavy and light chains. The full length TTx protein has the amino acid sequence set forth in SEQ ID NO: 26 (UniProt accession number P04958), and the TTx light chain has the amino acid sequence set forth in SEQ ID NO: 27. Thus in this embodiment the spacer has a sequence which is not present in either the heavy or light TTx chains, i.e. it is not present in SEQ ID NO: 22 or SEQ ID NO: 27.

In one embodiment of the invention, the spacer sequence comprises or consists of the amino acid sequence set forth in SEQ ID NO: 28, SEQ ID NO: 99 or SEQ ID NO: 102, or an amino acid sequence with at least 70, 75, 80, 85, 90 or 95% sequence identity thereto.

In one embodiment of the invention, the B-cell epitope-containing peptide contains a cysteine residue. In further embodiments the B-cell epitope-containing peptide comprises only one cysteine residue. The cysteine residue may be located within the MTTE, or within a spacer, or at the N- or C-terminus of the B-cell epitope-containing peptide. The cysteine residue may be used to conjugate the B-cell epitope-containing peptide to the T-cell epitope-containing antigen. In one embodiment of the invention the cysteine residue is located at the C-terminus of the B-cell epitope-containing peptide. For instance, the B-cell epitope-containing peptide may comprise or consist of a peptide consisting of, from N-terminus to C-terminus, an MTTE with SEQ ID NO: 1, a spacer with SEQ ID NO: 28 or SEQ ID NO: 99, and a cysteine residue. Such a B-cell epitope-containing peptide has the sequence set forth in SEQ ID NO: 21 or SEQ ID NO: 100, respectively (the B-cell epitope-containing peptide of SEQ ID NO: 21 comprises, from N-terminus to C-terminus, an MTTE of SEQ ID NO: 1 and a spacer with SEQ ID NO: 102), and the B-cell epitope-containing peptide of the conjugates of the invention thus may comprise or consist of the amino acid sequence of SEQ ID NO: 21 or SEQ ID NO: 100, or an amino acid sequence with at least 70, 75, 80, 85, 90, 95, 96, 97, 98 or 99% sequence identity thereto.

The B-cell epitope-containing peptide may thus be conjugated to the T-cell epitope-containing antigen via a thiol group emanating from the B-cell epitope-containing peptide. In particular embodiments, as detailed above, the thiol group is the side chain thiol group of a cysteine residue. However, the thiol group may be provided otherwise than on a cysteine residue. Indeed, it is not essential that the B-cell epitope-containing peptide contains a cysteine residue at all. The thiol group may be provided by any compound which contains such a group. The B-cell epitope-containing peptide may be conjugated to a thiol group-containing molecule. The conjugation of the peptide to the molecule must occur by a means which leaves a free thiol group in the resulting conjugate, so that the free thiol group can be used to conjugate the B-cell epitope-containing peptide to the T-cell epitope-containing antigen. Where a B-cell epitope-containing peptide is conjugated to a thiol group-containing molecule, the molecule is preferably conjugated to the N-terminal or C-terminal amino acid of the peptide.

The B-cell epitope-containing peptide may be synthesised by any method known in the art, such as using a protein expression system, or by chemical synthesis in a non-biological system, e.g. by liquid-phase synthesis or solid-phase synthesis.

The B-cell epitope-containing peptide may be conjugated to the T-cell epitope-containing antigen via any method known in the art. For instance, conjugation of the B-cell epitope-containing peptide to the T-cell epitope-containing antigen, may be via the N-terminal amino group of the B-cell epitope-containing peptide, or via its C-terminal carboxyl group, or via any reactive side-chain group. For instance, the conjugation may be via a hydroxyl group of a serine or threonine, the carboxyl group of an aspartate or glutamate, or the ε-amino group of a lysine. The conjugation may alternatively be via the thiol group of a cysteine residue located within the B-cell epitope-containing peptide. Alternatively, the B-cell epitope-containing peptide may be conjugated to the T-cell epitope-containing antigen by non-covalent interactions.

The B-cell epitope-containing peptide may be conjugated to the T-cell epitope-containing antigen directly or indirectly. As will be described in more detail below the B-cell epitope-containing peptide may be conjugated to the T-cell epitope-containing antigen directly via a covalent or non-covalent bond, such as a peptide bond, or it may be conjugated indirectly via a linking group or moiety. This may be a peptide-based linker group (i.e. a peptide sequence) or it may be a non-peptide based linker moiety or group.

In one embodiment, a conjugate of the invention comprises at least one B-cell epitope-containing peptide as defined herein, such as 1, 2, 3, 4, 5, 6, 7, 8, 9, 10, 11, 12, 13, 14, or 15 or 20 B-cell epitope-containing peptides. In further embodiments a conjugate of the invention comprises at most 50, 40, 30, 25, 20, 15 or 10 B-cell epitope-containing peptides as defined herein. In yet another embodiment, a conjugate of the invention comprises at least two B-cell epitope-containing peptides as defined herein, or at least three B-cell epitope-containing peptides as defined herein. In one embodiment, a conjugate of the invention comprises three B-cell epitope-containing peptides.

Conjugation of the one or more B-cell epitope-containing peptides to the T-cell epitope-containing antigen according to the invention may be via the N-terminal amino acid of the T-cell epitope-containing antigen, e.g. via a side-chain group of the N-terminal amino acid of the T-cell epitope-containing antigen or the N-terminal amino group of the T-cell epitope-containing antigen. Alternatively, conjugation of the one or more B-cell epitope-containing peptides to the T-cell epitope-containing antigen may be via the C-terminal amino acid of the T-cell epitope-containing antigen, e.g. via a side chain group of the C-terminal amino acid of the T-cell epitope-containing antigen or the C-terminal carboxyl group of the T-cell epitope-containing antigen.

One embodiment of the invention is a conjugate comprising one B-cell epitope-containing peptide, wherein the B-cell epitope-containing peptide is conjugated directly to the T-cell epitope-containing antigen via a peptide bond between the C-terminus of the B-cell epitope-containing peptide and the N-terminus of the T-cell epitope-containing antigen, i.e. the conjugate may consist of a single peptide chain comprising both the B-cell epitope-containing peptide and the T-cell epitope-containing antigen. Alternatively, the B-cell epitope-containing peptide and the T-cell epitope-containing antigen may be joined via a peptide linker.

Methods for conjugating a B-cell epitope-containing peptide to a T-cell epitope-containing antigen (or a vehicle comprising the T-cell epitope-containing antigen) are known in the art, such as in Hermanson (1996), Bioconjugate Techniques, Academic Press; U.S. Pat. Nos. 6,180,084; and 6,264,914, and include e.g. methods used to link haptens to carrier proteins as routinely used in applied immunology (see Harlow & Lane (1988), “Antibodies: A Laboratory Manual”, Cold Spring Harbor Laboratory Press, Cold Spring Harbor, N.Y., herein incorporated by reference).

A linker moiety may be used to conjugate the B-cell epitope-containing peptide to the T-cell epitope-containing antigen. A linker moiety may be provided in the form of a chemical moiety, or compound, which comprises reactive, or functional groups, for reaction with respective, or cognate, functional or reactive groups provided on or in the respective B-cell epitope-containing peptide and T-cell epitope-containing antigen. Such a linker moiety may be regarded as a core compound to which the peptide(s) and antigen respectively are linked or coupled to form the conjugate. In one embodiment of the invention, a conjugate comprises a core compound (or linker moiety) linked to (i) at least one B-cell epitope-containing peptide and to (ii) a T-cell epitope-containing peptide.

In one embodiment of the invention, the B-cell epitope-containing peptide of a conjugate of the invention is conjugated to a T-cell epitope-containing antigen via a peptide linker.

In another embodiment the linker moiety (e.g. core compound) may contain maleimide groups for conjugation (i.e. linkage) to thiol groups present in the peptide(s) and/or antigen. The thiol groups may be present in the B-cell epitope-containing peptide(s), which are accordingly linked to the core compound in the conjugates via succinimide groups. In other embodiments the linker moiety (e.g. core compound) may contain other (i.e. any) functional or reactive groups capable of reacting with functional or reactive groups present in the peptide(s) and antigen to be conjugated. Thus a linker moiety (e.g. core compound) may contain two or more chemical groups which are reactive with chemical groups present in or on the peptide(s) and antigen to be conjugated. Such chemical groups present in or on the peptide(s) and antigen may be termed cognate chemical groups (or cognate reactive/functional groups).

In an embodiment a chemical/reactive/functional group in the core compound which is reactive with a chemical/reactive/functional group present in the B-cell epitope-containing peptide is different to the chemical/reactive/functional group in the core compound which is reactive with a chemical/reactive/functional group present in the T-cell epitope-containing antigen, and the cognate chemical/reactive/functional groups present in the peptide(s) and antigen respectively are different. A wide range of different reactive (or functional) groups and coupling chemistries upon which such reactive/functional groups may be based are known in the art and reported in the literature and any such reactive group (or alternatively termed, reactive moiety, or functional group or functional moiety) may be used.

In an embodiment the reactive group (for example the reactive group which is reactive with the T-cell epitope-containing antigen) is or comprises an alkyne group, for example a cycloalkyne group. The cycloalkyne group may be e.g. a C5-C10 cycloalkyne group such as a cyclooctyne group. In one embodiment the reactive group may be or may comprise a diphenylycycloctyne group. An alkyne reactive group may be reactive with an azido group provided in or on the peptide or antigen to be conjugated (e.g. in or on the T-cell epitope-containing antigen). In an embodiment, the B-cell epitope-containing peptide(s) are coupled to a compound core by a thiol-maleimide linkage between thiol groups in the peptide(s) and maleimide group(s) in the core compound (i.e. via a succinimide group) and the T-cell epitope-containing antigen is coupled to the compound core by a linkage between an azido group present in the antigen and an alkyne group in the core compound.

An azido group may be introduced into a peptide, such as into the T-cell epitope-containing antigen, for example at the N-terminus thereof, by any means known in the art. Thus an azido group-containing moiety may be coupled to the antigen, e.g. to the N-terminal amino acid thereof. For example, an azidocarboxylic acid group may be introduced at the N-terminus, e.g. an azido-C2-C8 carboxylic acid, e.g. an azidohexaenoyl or azidopropanoyl group. This may be achieved by reacting the antigen with an azidocarboxylic acid to couple the azidocarboxylic acid to the N-terminal amino acid of the antigen, for example by means of an amide bond between the N-terminal amino group of the antigen and the carboxylic acid group of the azidocarboxylic acid. Alternatively, an amino acid derivative comprising an azido group in the side chain may be introduced into the antigen during peptide synthesis of the antigen, and may be present at any position in the antigen peptide chain.

A linker may be, or comprise, or be based on or derived from, tri-amino-2,2-dimethyl propanoic acid with a diphenylcyclooctyne PEG spacer. This has the structure set forth in Formula I:

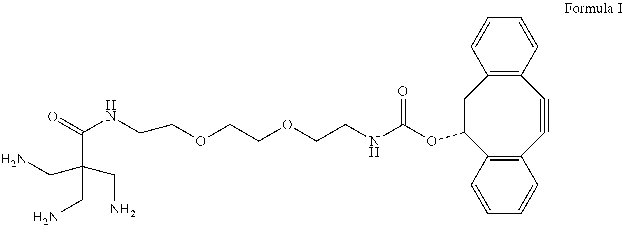

The amino groups in an intermediate compound of Formula I may be protected by protecting groups, e.g. Boc (tert-butyloxycarbonyl) groups. Such protecting groups may be removed before subsequent reaction.

In an embodiment, the amino groups of the compound of Formula I may be functionalised with propionyl maleimide, which has the structure set forth in Formula II, to yield a functionalised linker with the structure set forth in Formula III. Such a functionalised linker may be regarded as a linker moiety or core compound, as discussed above.

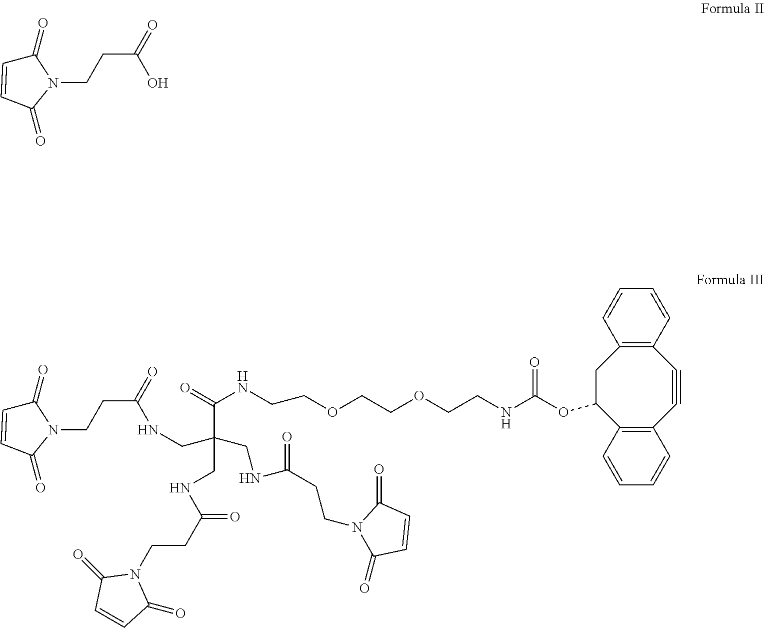

Three B-cell epitope-containing peptides (BCECPs) can be conjugated to the structure of Formula III via thiol groups to the maleimide groups. Conjugation of thiol groups to maleimide groups is common in the art, and occurs by Michael addition of a thiolate to the maleimide double bond to form a succinimidyl thioether (SITE).

The T-cell epitope-containing antigen (TCECA) may first be conjugated to hexanoyl azide (Formula IV):

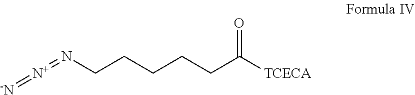

In the structure of Formula IV the T-cell epitope-containing antigen may be directly bound to the azidohexanoic acid group via an amide bond formed between the N-terminal amino group of the T-cell epitope-containing antigen and the carboxyl group of the azidohexanoic acid. However, as noted above, an azido group may alternatively be introduced as part of the side chain of a derivatised amino acid introduced at any position of the peptide chain of the antigen. The azidohexanoyl antigen of Formula IV, or any azido group-containing antigen, can be conjugated to the linker at the location of the carbon-carbon triple bond. The resultant structure is shown in Formula V:

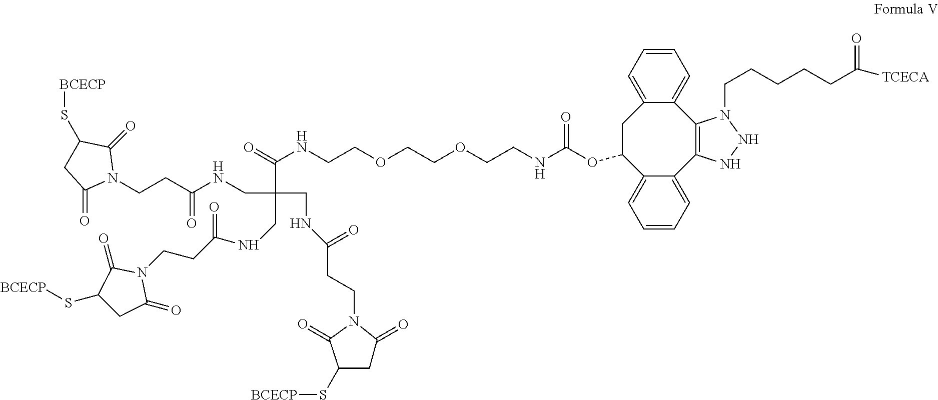

One embodiment of the invention is a conjugate of the structure presented in Formula V, wherein each of the three sulphur atoms is the sulphur of a thiol group of a cysteine residue of the relevant B-cell epitope-containing peptide. Methods for making such conjugates are taught in WO 2011/115483.

Other embodiments of the invention are conjugates of the structure of Formula VI or Formula VII:

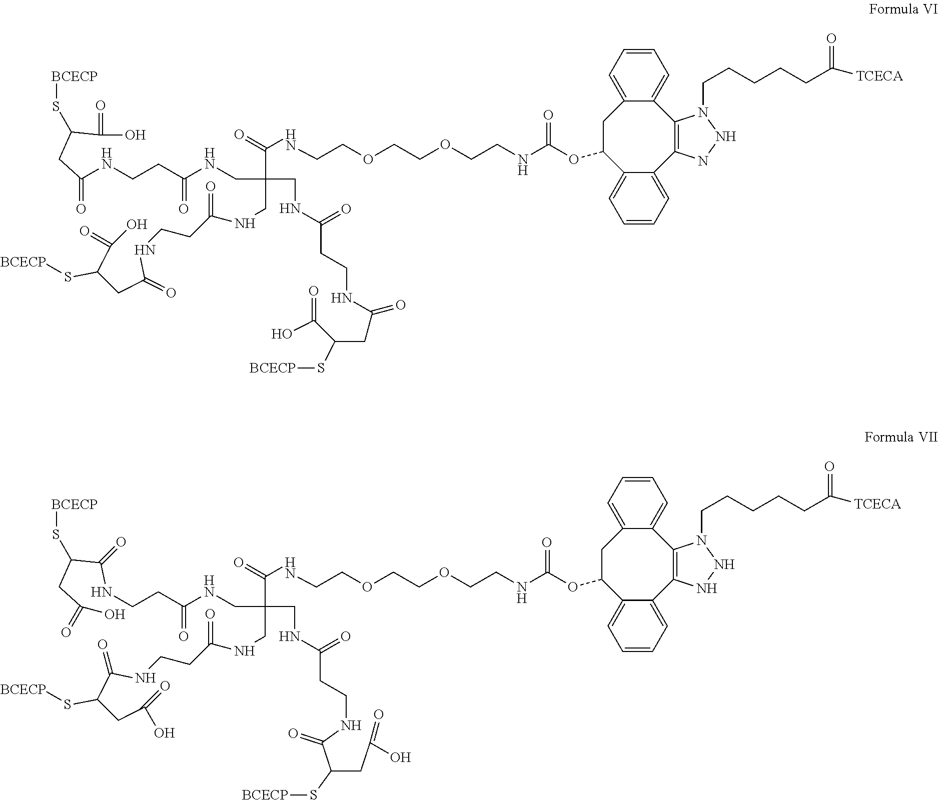

A conjugate of Formula VI or Formula VII may be obtained by ring opening of a conjugate of Formula V.

As will be known and apparent to the skilled person, hydrolysis of the succinimide ring of a SITE yields an isomeric succinamic acid thioether (SATE). Also within the scope of the invention are structural isomers, enantiomers, diastereomers and stereoisomers of a conjugate of Formula VI, or structural isomers, enantiomers, diastereomers and stereoisomers of a conjugate of Formula VII. Any variations in arrangement or stereochemistry and/or combinations thereof of the conjugates of Formula VI and Formula VII are within the scope of the invention.

Hydrolysis of the succinimide ring of a SITE may occur spontaneously under appropriate conditions (see e.g. Fontaine et al., supra). Ring opening may be performed in an aqueous solution at pH 5 or above, for instance about pH 6 (e.g. between pH 5.5 and pH 6.5), or at pH 7 or above or pH 8 or above. The solution may contain additional solvents, such as solvents which enhance solubility of the conjugate, for instance acetonitrile or tert-butanol. The solution may be buffered, for instance a carbonate buffer (e.g. sodium bicarbonate) may be used to maintain the desired pH. Ring opening may be performed at a temperature above room temperature, for example at least 25, 30, 40, or 50° C. or more, e.g. 25 to 35° C. or about 30° C. For instance, ring opening may be performed at about 30° C., at a pH of about 6 in a solution comprising acetonitrile and tert-butanol and an NaHCO₃ buffer. Ring opening occurs after conjugation of the B-cell epitope-containing peptides to the linker core, but may occur before or after conjugation of the T-cell epitope-containing antigen to the linker core. Following synthesis, the conjugate may be purified by any method known in the art, e.g. by using HPLC.

A Type A conjugate of the present invention thus comprises an antigen containing a CD8+ T-cell cancer epitope N-terminal to a CD4+ T-cell cancer epitope.

A CD8+ T-cell cancer epitope is an epitope presented by a Class I MHC (MHC I) molecule; a CD4+ T-cell cancer epitope is presented by a Class II MHC (MHC II) molecule. CD8+ T-cells recognise antigen-MHC I complexes, while CD4+ T-cells recognise antigen-MHC II complexes. As is known to the skilled person, MHC I molecules are expressed by essentially all nucleated cells, while MHC II molecules are generally expressed by professional antigen-presenting cells (APCs) and activated T-cells along with some tumour cells. APCs include in particular dendritic cells, macrophages and B-cells, though other cell types may also be considered APCs. MHC I molecules primarily present peptides generated by degradation of cytosolic/intracellular proteins; MHC II molecules present peptides generated by degradation of exogenous proteins. MHC I molecules' primarily function is to present epitopes from intracellular pathogens (e.g. viruses) and epitopes produced by mutation of native genes (e.g. cancer antigens). MHC II molecules primarily function to present epitopes from extracellular pathogens and/or toxins etc., e.g. bacterial or parasitic infections. Some APCs (e.g. dendritic cells) are able to present peptides generated by degradation of exogenous proteins on MHC I molecules, in a process known as cross-presentation. Cross presentation is important in the activation of CD8+ cells to fight intracellular infections which do not generally infect APCs, and also to attack tumour cells etc. which produce antigens not found in healthy APCs.

CD8+ T-cells are also known as cytotoxic T-cells (CTLs). When a CD8+ T-cell epitope being presented by an MHC I is recognised by a CTL, the response of the CTL is to release cytotoxins which kill the target cell. Conversely, when a CD4+ T-cell epitope is recognised by a CD4+ T-cell (a helper T-cell), the CD4+ T-cell is activated to support immune responses by other parts of the immune system.

The CD8+ T-cell cancer epitope according to the invention may be any cancer-derived peptide which, when presented by an MHC 1, is recognised by a CD8+ T-cell. There is no limitation to the sequence or length of the peptide so long as it is recognisable by a CD8+ T-cell. Commonly the peptide is 9-10 amino acids long, but can be between 8-15 amino acids long in some cases. Similarly, the CD4+ T-cell cancer epitope according to the invention may be any cancer-derived peptide which, when presented by an MHC 1, is recognised by a CD4+ T-cell. There is no limitation to the sequence or length of the peptide so long as it is recognisable by a CD4+ T-cell. Commonly it is at least 11 amino acids long and can be up to 30 amino acids long.

CD8+ T-cell epitopes are generally 8-10 amino acids in length, though this may vary. A CD8+ T-cell cancer epitope as defined herein may be from 8-15 amino acids in length. A CD4+ T-cell cancer epitope as defined herein may be from 11-30 amino acids in length. As used herein, a T-cell epitope-containing antigen may be from 15-50 or 20-50 amino acids long. In embodiments of the invention, the antigen may be at least 15, 20, 25, 26, 27 or 30 amino acids long. In other, non-limiting, embodiments the antigen may be at most 100, 90, 80, 70, 60, 50, 45, 40, 35 or 34 amino acids long. For instance, the T-cell epitope-containing antigen may be from 15-40 amino acids long, such as 20-40, 25-35 or 28-34 amino acids long.

T-cell epitopes may be identified experimentally, e.g. by T-cell epitope mapping, methods for which are known in the art (e.g. flow cytometry, see Kern et al. (1998), Nat Med 4: 975-978). T-cell epitopes can also be predicted using bioinformatic approaches (see e.g. Desai & Kulkarni-Kale (2014), Methods Mol. Biol. 1184:333-364). A T-cell epitope as defined herein may be identified by any method known in the art.

The T-cell epitope-containing antigen comprises a CD8+ T-cell cancer epitope N-terminal to a CD4+ T-cell cancer epitope. The epitopes may be directly or indirectly linked. Thus, the CD8+ T-cell cancer epitope may be immediately N-terminal to the CD4+ T-cell cancer epitope, i.e. the epitopes may be directly adjacent with no intervening amino acids between the C-terminal amino acid of the CD8+ T-cell cancer epitope and the N-terminal amino acid of the CD4+ T-cell cancer epitope. Alternatively, the two epitopes may be separated by a spacer of at least one amino acid. Such a spacer may be of any length, e.g. 1-10 amino acids, for instance 1-9, 1-8, 1-7 or 1-6 amino acids, e.g. 1, 2, 3, 4, 5 or 6 amino acids.

The cancer-related epitope may be an epitope from a wild-type protein associated with cancer, e.g. a protein commonly overexpressed in cancer or in certain cancers. Examples of many such cancer-associated proteins are known in the art.

In an embodiment of the invention, at least one of the CD8+ T-cell cancer epitope(s) and CD4+ T-cell cancer epitope(s) is derived from a protein associated with prostate cancer. In a further aspect of the invention, the CD8+ and the CD4+ T-cell cancer epitopes are derived from a protein associated with prostate cancer. In this case, the CD8+ and CD4+ T-cell cancer epitopes may be derived from the same prostate cancer-associated protein, or from different prostate cancer-associated proteins.

An example of a prostate cancer-associated protein, from which the CD8+ and/or CD4+ T-cell cancer epitopes may be derived, is glutamate carboxypeptidase 2 (GCPII). GCPII has the UniProt accession number Q04609, and is encoded by the gene FOLH1. The amino acid sequence of human GCPII is presented in SEQ ID NO: 24.

Another example of a prostate cancer-associated protein is prostatic acid phosphatase (PAP). PAP has the UniProt accession number P15309, and is encoded by the gene ACPP. The amino acid sequence of human PAP is presented in SEQ ID NO: 25.

In one embodiment of the invention, the CD8+ T-cell cancer epitope and/or the CD4+ T-cell cancer epitope is derived from human GCPII (i.e. from SEQ ID NO: 24) or from human PAP (i.e. SEQ ID NO: 25). The CD8+ T-cell epitope may comprise or consist of an 8-15 amino acid fragment of SEQ ID NO: 24 or SEQ ID NO: 25, or an amino acid sequence with at least 65, 70, 75, 80, 85, 90 or 95% sequence identity to such a fragment. The wording “fragment” as used herein refers to a sequence of amino acids which are contiguous in SEQ ID NO: 24 or SEQ ID NO: 25.

The fragment of SEQ ID NO: 24 or SEQ ID NO: 25 which forms or is found within the CD8+ T-cell cancer epitope (or a variant of which forms or is found within the CD8+ T-cell cancer epitope) may be 8-15 amino acids long, e.g. 8-10 amino acids, or 8, 9, 10, 11, 12, 13, 14 or 15 amino acids long. In one embodiment, the fragment is 9-10 amino acids long. The CD8+ T-cell cancer epitope may comprise or consist of an 8-10 amino acid fragment of SEQ ID NO: 24 or of SEQ ID NO: 25, or an amino acid sequence with at least 65, 70, 75, 80, 85, 90 or 95% sequence identity to any such fragment. In one embodiment, the fragment is 9 amino acids long, i.e. the CD8+ T-cell cancer epitope comprises or consists of a 9 amino acid fragment of SEQ ID NO: 24 or of SEQ ID NO: 25, or an amino acid sequence with at least 65, 70, 75, 80, 85, 90 or 95% sequence identity to any such fragment. The fragment may be located at any position within SEQ ID NO: 24 or SEQ ID NO: 25.

The CD4+ T-cell cancer epitope may comprise or consist of an 11-30 amino acid fragment of SEQ ID NO: 24 or SEQ ID NO: 25, or an amino acid sequence with at least 75, 80, 85, 90 or 95% sequence identity to such a fragment. In an embodiment, the CD4+ T-cell cancer epitope comprises or consists of a sequence of 11-20 amino acids which are contiguous in SEQ ID NO: 24 or SEQ ID NO: 25, or a sequence of amino acids which has at least 75, 80, 85, 90 or 95% sequence identity to a sequence of 11-20 amino acids which are contiguous in SEQ ID NO: 24 or SEQ ID NO: 25.

The fragment of SEQ ID NO: 24 or SEQ ID NO: 25 which forms or is found within the CD4+ T-cell cancer epitope (or a variant of which forms or is found within the CD4+ T-cell cancer epitope) may be 11-20 amino acids long, e.g. 11-18, 12-18, 10-15, 12-15, 12-16 or 14-16 amino acids, or 11, 12, 13, 14, 15, 16, 17 or 18 amino acids long. In one embodiment, the fragment is 12-18 amino acids long. The CD8+ T-cell cancer epitope may comprise or consist of a 12-18 amino acid fragment of SEQ ID NO: 24 or of SEQ ID NO: 25, or an amino acid sequence with at least 75, 80, 85, 90 or 95% sequence identity to any such fragment. In one embodiment, the fragment is 15 amino acids long, i.e. the CD4+ T-cell cancer epitope may comprise or consist of a 15 amino acid fragment of SEQ ID NO: 24 or of SEQ ID NO: 25, or an amino acid sequence with at least 75, 80, 85, 90 or 95% sequence identity to any such fragment. The fragment may be located at any position within SEQ ID NO: 24 or SEQ ID NO: 25.

In certain embodiments of the invention, the CD8+ T-cell cancer epitope is selected from any one of SEQ ID NOs: 2, 3, 4, 5 and 6, or is an amino acid sequence with at least 65, 70, 75, 80, 85, 90 or 95% sequence identity to SEQ ID NO: 2, 3, 4, 5 or 6. SEQ ID NO: 2 is derived from GCPII and corresponds to amino acids 178-186 of SEQ ID NO: 24; SEQ ID NO: 3 is derived from GCPII and corresponds to amino acids 4-12 of SEQ ID NO: 24; SEQ ID NO: 4 is derived from PAP and corresponds to amino acids 13-21 of SEQ ID NO: 25; SEQ ID NO: 5 is derived from GCPII and corresponds to amino acids 168-176 of SEQ ID NO: 24; SEQ ID NO: 6 is derived from GCPII and corresponds to amino acids 207-215 of SEQ ID NO: 24.

In other embodiments of the invention, the CD4+ T-cell cancer epitope is selected from any one of SEQ ID NOs: 7, 8, 9, 10 and 11, or is an amino acid sequence with at least 75, 80, 85, 90 or 95% sequence identity to SEQ ID NO: 7, 8, 9, 10 or 11. SEQ ID NO: 7 is derived from PAP and corresponds to amino acids 199-213 of SEQ ID NO: 25; SEQ ID NO: 8 is derived from GCPII and corresponds to amino acids 730-744 of SEQ ID NO: 24; SEQ ID NO: 9 is derived from GCPII and corresponds to amino acids 206-220 of SEQ ID NO: 24; SEQ ID NO: 10 is derived from GCPII and corresponds to amino acids 334-348 of SEQ ID NO: 24; SEQ ID NO: 11 is derived from GCPII and corresponds to amino acids 459-473 of SEQ ID NO: 24.

The T-cell epitopes disclosed above are known from the literature as prostate cancer epitopes: the CD8+ T-cell cancer epitopes of SEQ ID NOs: 2 and 6 were identified in Kiessling et al. (2008), Eur. Urol. 53: 694-708; that of SEQ ID NO: 3 in Matera (2010), Cancer Treat. Rev. 36: 131-141, and those of SEQ ID NO: 4 in US 2006/0263342 and SEQ ID NO: 5 in US 2005/0260234; the CD4+ T-cell epitope of SEQ ID NO: 7 was identified in McNeel et al. (2001), Cancer Res. 61: 5161-5167; that of SEQ ID NO: 9 was identified in Younger et al (2008), Prostate Cancer Prostatic Dis. 11: 334-341; those of SEQ ID NOs: 8 and 11 were identified in Schroers et al. (2003), Clin. Cancer Res. 9: 3260-3271; and that of SEQ ID NO: 10 was identified in Kobayashi et al. (2003), Clin. Cancer Res. 9: 5386-5393.

These T-cell epitopes are specifically recognised by a variety of human HLA-A types. The CD8+ T-cell epitope of SEQ ID NO: 2 is recognised by at least HLA-A24 (also known as HLA-A*24), those of SEQ ID NOs: 3-4 are recognised by at least HLA-A2 (also known as HLA-A*02), that of SEQ ID NO: 5 is recognised by at least HLA-A1 (also known as HLA-A*01) and that of SEQ ID NO: 6 is recognised by at least HLA-A3, HLA-A11, HLA-A31 and HLA-A33 (also known as HLA-A*03, HLA-A*11, HLA-A*31 and HLA-A*33, respectively). The MHC class II-epitope interaction is more promiscuous, and SEQ ID NOs: 7, 8, 9, 10 and 11 can bind to various HLA-DR alpha/beta heterodimers (and binding may not be limited to HLA-DR).

Where a T-cell epitope of the invention has less than 100% sequence identity to those defined herein (i.e. it is a variant T-cell epitope), it is necessary that the epitope be a functional epitope variant recognised by a TCR which also recognises the native sequence, in order to stimulate an immune response against the native antigen. This can be determined using functional assays known in the art, e.g. assays which measure T-cell activation based on their production of cytokines such as IFNγ and TNF in response to a stimulus. For a variant epitope sequence to be considered a functional variant epitope, at least 50, 60, 70, 80, 90 or 95% of T-cells which recognise it should also recognise the native epitope sequence. Most preferably, all T-cells which recognise the variant epitope sequence also recognise the native epitope sequence.

The T-cell epitope-containing antigen in a conjugate of the invention may comprise a protease recognition site (i.e. a site recognised and cleaved by a protease, also known as a protease cleavage site) between the CD8+ T-cell cancer epitope and the CD4+ T-cell cancer epitope. Any known protease recognition site may be used if it is suitable for marking the T-cell epitope-containing antigen for cleavage between the two epitopes. The recognition site may be for any cytosolic or endoplasmic reticulum (ER) protease: that is, any protease found within the cytosol or ER of a human cell. In one embodiment, the protease recognition site is a proteasome recognition site (or cleavage site).

Proteasome cleavage sites may be predicted using appropriate computer programmes and software, e.g. the online programme NetChop (Nielsen et al. (2005), Immunogenetics 57(1-2): 33-41), accessible at http://www.cbs.dtu.dk/services/NetChop. For proper presentation of CD8+ T-cell cancer epitopes by MHC I complexes, the C-terminus of the epitope must be properly generated by the proteasome (particularly the immunoproteasome).

A proteasome cleavage site may be located between the CD8+ T-cell cancer epitope and the CD4+ T-cell cancer epitope of the T-cell epitope-containing antigen. The proteasome cleavage site may be located directly between the CD8+ T-cell cancer epitope and the CD4+ T-cell cancer epitope, i.e. in this embodiment no additional amino acids are present between the CD8+ T-cell epitope and the CD4+ T-cell epitope, which are joined directly to one another by a peptide bond between the C-terminal amino acid of the CD8+ T-cell epitope and the N-terminal amino acid of the CD4+ T-cell epitope. Alternatively, the proteasome cleavage site may be provided by additional amino acids located between the CD8+ T-cell epitope and the CD4+ T-cell epitope, i.e. in this embodiment the C-terminal amino acid of the CD8+ T-cell cancer epitope is separated from the N-terminal amino acid of the CD4+ T-cell cancer epitope by a number of additional amino acids to form the designed cleavage site for proper epitope processing in vivo.

When the proteasome cleavage site is provided by additional amino acids located between the CD8+ and CD4+ T-cell epitopes (i.e. an amino acid spacer), the spacer may be any number of amino acids long. In one embodiment of the invention, the spacer is no more than 6 amino acids long, such as 1, 2, 3, 4, 5 or 6 amino acids long. The spacer may be any amino acid sequence, but is a sequence which provides a proteasome cleavage site between the two epitopes. The sequence of the spacer will therefore be dependent on the sequences of the flanking epitopes.

When the proteasome cleavage site is provided by a spacer, the proteasome cleavage site may be located within the spacer, i.e. the proteasome may cleave the T-cell epitope-containing antigen between two amino acids of the spacer, such that residues of the spacer remain on the C-terminus of the CD8+ T-cell cancer epitope and the N-terminus of the CD4+ T-cell cancer epitope following antigen cleavage. In one embodiment of the invention, the cleavage site provided by the spacer is located between the N-terminal residue of the spacer and C-terminal amino acid of the CD8+ T-cell cancer epitope, such that following antigen cleavage spacer residues remain only on the N-terminus of the CD4+ T-cell cancer epitope, and none on the CD8+ T-cell cancer epitope.

The T-cell epitope-containing antigen forming part of a conjugate of the invention comprises a translocation peptide positioned N-terminal to the CD8+ T-cell cancer epitope. In one aspect of the invention, the translocation peptide mediates TAP-driven transport of the T-cell epitope-containing antigen, or at least the CD8+ T-cell cancer epitope located therein, into the endoplasmic reticulum of a host cell.

In one aspect of the invention, the translocation peptide is a short sequence of amino acids which are recognised by the TAP complex and form the N-terminus of the translocated peptide, such as a peptide which is 3-5 amino acids long, e.g. 3, 4 or 5 amino acids long. In yet another aspect of the invention, the translocation peptide is a peptide mediating TAP-driven transport of at least the CD8+ T-cell cancer epitope. In one embodiment of the invention, the translocation peptide has the amino acid sequence ARWW (SEQ ID NO: 12), or an amino acid sequence with at least 75 or 80% sequence identity thereto.

In an embodiment, the translocation peptide and the CD8+ T-cell cancer epitope are directly adjacent to each other in the T-cell epitope-containing antigen (i.e. the C-terminal amino acid of the translocation peptide is directly N-terminal to the N-terminal amino acid of CD8+ T-cell cancer epitope, such that these two amino acids are joined by a peptide bond).

In one embodiment of the invention, the translocation peptide forms the N-terminus of the T-cell epitope-containing antigen, directly C-terminal to which is the CD8+ T-cell cancer epitope, that is in turn directly N-terminal to either the CD4+ T-cell cancer epitope or a spacer immediately followed by a CD4+ T-cell cancer epitope. The presence of a proteasome cleavage site between the epitopes allows separation of the epitopes, such that a fragment is produced consisting of the translocation peptide and the CD8+ T-cell cancer epitope, which fragment is of a length allowing for TAP-driven translocation.

A peptide able to mediate TAP-driven translocation may be identified experimentally by TAP translocation assay. TAP translocation assays are described in detail in Jongsma & Neefjes (2013), Antigen Processing: Methods and Protocols (edited by Peter van Endert), Chapter 5 (p53-65).

In one embodiment of the invention, the conjugate comprises a T-cell epitope-containing antigen containing a CD8+ T-cell epitope comprising or consisting of the sequence set forth in SEQ ID NO: 2, or an amino acid sequence with at least 65, 70, 75, 80, 85, 90 or 95% sequence identity thereto; and a CD4+ T-cell epitope comprising or consisting of the sequence set forth in SEQ ID NO: 7, or an amino acid sequence with at least 75, 80, 85, 90 or 95% sequence identity thereto (Conjugate I.).

In one embodiment of the invention, the T-cell epitope-containing antigen of Conjugate I comprises a translocation peptide with the sequence set forth in SEQ ID NO: 12, and a spacer with the sequence QQQPPP (SEQ ID NO: 29) separating the two T-cell epitopes. The T-cell epitope-containing antigen of Conjugate I may comprise or consist of the amino acid sequence set forth in SEQ ID NO: 13, or an amino acid sequence with at least 70, 75, 80, 85, 90 or 95% sequence identity thereto.

In another embodiment of the invention, the conjugate comprises a T-cell epitope-containing antigen containing a CD8+ T-cell cancer epitope comprising or consisting of the sequence set forth in SEQ ID NO: 3, or an amino acid sequence with at least 65, 70, 75, 80, 85, 90 or 95% sequence identity thereto; and a CD4+ T-cell cancer epitope comprising or consisting of the sequence set forth in SEQ ID NO: 8, or an amino acid sequence with at least 75, 80, 85, 90 or 95% sequence identity thereto (Conjugate II).

In one embodiment of the invention, the T-cell epitope-containing antigen of Conjugate II comprises a translocation peptide with the sequence set forth in SEQ ID NO: 12, and a spacer with the sequence AAA, separating the two T-cell epitopes. The T-cell epitope-containing antigen of Conjugate II may comprise or consist of the amino acid sequence set forth in SEQ ID NO: 14, or an amino acid sequence with at least 70, 75, 80, 85, 90 or 95% sequence identity thereto.

In yet another embodiment of the invention, the conjugate comprises a T-cell epitope-containing antigen containing a CD8+ T-cell cancer epitope comprising or consisting of the sequence set forth in SEQ ID NO: 4, or an amino acid sequence with at least 65, 70, 75, 80, 85, 90 or 95% sequence identity thereto; and a CD4+ T-cell cancer epitope comprising or consisting of the sequence set forth in SEQ ID NO: 9, or an amino acid sequence with at least 75, 80, 85, 90 or 95% sequence identity thereto (Conjugate III).

In one embodiment of the invention, the T-cell epitope-containing antigen of Conjugate III comprises a translocation peptide with the sequence set forth in SEQ ID NO: 12, and a spacer with the sequence AAA separating the two T-cell epitopes. The T-cell epitope-containing antigen of Conjugate III may comprise or consist of the amino acid sequence set forth in SEQ ID NO: 15, or an amino acid sequence with at least 70, 75, 80, 85, 90 or 95% sequence identity thereto.

In yet another embodiment of the invention, the conjugate comprises a T-cell epitope-containing antigen containing a CD8+ T-cell cancer epitope comprising or consisting of the sequence set forth in SEQ ID NO: 5, or an amino acid sequence with at least 65, 70, 75, 80, 85, 90 or 95% sequence identity thereto; and a CD4+ T-cell cancer epitope comprising or consisting of the sequence set forth in SEQ ID NO: 10, or an amino acid sequence with at least 75, 80, 85, 90 or 95% sequence identity thereto (Conjugate IV).

In one embodiment of the invention, the T-cell epitope-containing antigen of Conjugate IV comprises a translocation peptide with the sequence set forth in SEQ ID NO: 12, wherein the CD8+ and CD4+ T-cell cancer epitopes are directly adjacent (i.e. are not separated by a spacer). The T-cell epitope-containing antigen of Conjugate IV may comprise or consist of the amino acid sequence set forth in SEQ ID NO: 16, or an amino acid sequence with at least 70, 75, 80, 85, 90 or 95% sequence identity thereto.

In yet another embodiment of the invention, the conjugate comprises a T-cell epitope-containing antigen containing a CD8+ T-cell cancer epitope comprising or consisting of the sequence set forth in SEQ ID NO: 6, or an amino acid sequence with at least 65, 70, 75, 80, 85, 90 or 95% sequence identity thereto; and a CD4+ T-cell cancer epitope comprising or consisting of the sequence set forth in SEQ ID NO: 11, or an amino acid sequence with at least 75, 80, 85, 90 or 95% sequence identity thereto (Conjugate V).

In one embodiment of the invention, the T-cell epitope-containing antigen of Conjugate V comprises a translocation peptide with the sequence set forth in SEQ ID NO: 12, wherein the CD8+ and CD4+ T-cell cancer epitopes are directly adjacent (i.e. are not separated by a spacer). The T-cell epitope-containing antigen of Conjugate V may comprise or consist of the amino acid sequence set forth in SEQ ID NO: 17, or an amino acid sequence with at least 70, 75, 80, 85, 90 or 95% sequence identity thereto.

As detailed in the Examples, antigens comprising all combinations of the CD8+ T-cell cancer epitopes of SEQ ID NOs: 2-6 and the CD4+ T-cell cancer epitopes of SEQ ID NOs: 7-11 were synthesised and degraded using a commercially available immunoproteasome. Degradation after 24 hrs was analysed by MALDI-TOF mass spectrometry (MALDI-TOF MS), and the sequences of SEQ ID NOs: 13-17 were found to be most optimally degraded of all combinations. These sequences were found to be cleaved most effectively to yield the desired T-cell epitopes which can thus be presented in MHC I and MHC II to the immune system.

Another aspect of the invention is a Type B conjugate. A Type B conjugate comprises a T-cell epitope-containing antigen derived from cancer/testis antigen 1 (NY-ESO-1). NY-ESO-1 is encoded by the CTAG1A gene and has the UniProt accession number P78358. The amino acid sequence of human NY-ESO-1 is set forth in SEQ ID NO: 18. NY-ESO-1 is a tumour antigen: expression of NY-ESO-1 occurs only in the testes in healthy individuals, and its expression outside of this context is associated with several cancers, particularly melanoma and multiple myeloma, but also prostate cancer. NY-ESO-1 expression has been identified in up to 30% of prostate cancer patients, and vaccination of patients with NY-ESO-1 peptides was found to slow cancer growth (Sonpavde et al. (2014), Invest. New Drugs 32(2): 235-242).

The T-cell epitope-containing antigen of the Type B conjugate comprises a 20-35 amino acid fragment of SEQ ID NO: 18, or an amino acid sequence with at least 70, 75, 80, 85, 90 or 95% sequence identity to any such fragment. (As above, a 20-35 amino acid fragment of SEQ ID NO: 18 is a sequence of from 20 to 35 amino acids which are contiguous in SEQ ID NO: 18.) The fragment of SEQ ID NO: 18 may be e.g. 20-30, 25-35 or 25-30 amino acids in length. A Type B conjugate of the invention is known as Conjugate VI. In an embodiment, the T-cell epitope-containing antigen of Conjugate VI is from 20-50 amino acids long in total, e.g. 20-45. 20-40, 20-35, 25-40, 25-35, 30-50, 35-50, 30-40 or 35-40. In one embodiment of the invention, the T-cell epitope-containing antigen of Conjugate VI is at most 50 amino acids long.

The NY-ESO-1 peptide may be processed into T cell epitopes presented on MHC molecules. such as the amino acid sequence set forth in SEQ ID NO: 19 (Gnjatic et al. (2000), Proc. Natl. Acad. Sci. U.S.A. 97(20): 10917-10922), or an amino acid sequence with at least 65, 70, 75, 80, 85, 90 or 95% sequence identity thereto. SEQ ID NO: 19 corresponds to amino acids 92-100 of NY-ESO-1 (i.e. amino acids 92-100 of SEQ ID NO: 18). The peptide of SEQ ID NO: 19 is recognised by HLA-Cw3. The sequence of SEQ ID NO: 19 (or the variant thereof) can be located at the N-terminus, the C-terminus or in the middle of the T-cell epitope-containing antigen.

In one embodiment of the invention, the T-cell epitope-containing antigen of Conjugate VI comprises the CD4+ T-cell cancer epitope of SEQ ID NO: 101 (Mandic et al. (2005), J. Immunol. 174: 1751-1759) or an amino acid sequence with at least 75, 80, 85, 90 or 95% sequence identity thereto. SEQ ID NO: 101 corresponds to amino acids 87-101 of NY-ESO-1 (i.e. amino acids 87-101 of SEQ ID NO: 18). The sequence of SEQ ID NO: 101 (or the variant thereof) can be located at the N-terminus, the C-terminus or in the middle of the T-cell epitope-containing antigen.

In yet another embodiment of the invention, the T-cell epitope-containing antigen of Conjugate VI comprises or consists of the amino acid sequence of SEQ ID NO: 20, or an amino acid sequence with at least 70, 75, 80, 85, 90 or 95% sequence identity to SEQ ID NO: 20. SEQ ID NO: 20 corresponds to amino acids 79-105 of NY-ESO-1 (i.e. amino acids 79-105 of SEQ ID NO: 18). When the T-cell epitope-containing antigen of Conjugate VI comprises or consists of a variant sequence of SEQ ID NO: 20 (or a variant sequence of SEQ ID NO: 19 or SEQ ID NO: 101 or a variant fragment of SEQ ID NO: 18), it must be equivalently immunogenic to the equivalent native sequence (i.e. it must be functionally equivalent). Methods by which functional equivalence of antigen sequences can be analysed are discussed above.

The T-cell epitope-containing antigen of Conjugate VI may comprise one or more CD8+ T-cell cancer epitopes, and/or one or more CD4+ T-cell cancer epitopes. It may comprise a translocation peptide as defined above, and/or one or more proteasome cleavage sites. However, there is no requirement that any of these features be present.

Another aspect of the invention is a Type C conjugate. A type C conjugate comprises at least one B-cell epitope-containing peptide conjugated to a T-cell epitope-containing antigen, wherein:

(i) said at least one B-cell epitope-containing peptide comprises a minimal tetanus toxoid epitope (MTTE), said MTTE comprising:
(a) an amino acid sequence of at least 10 amino acids which are contiguous in SEQ ID NO: 22 and comprise the amino acid sequence GITELKKL set forth in SEQ ID NO: 23; or
(b) an amino acid sequence with at least 70% sequence identity to an amino acid sequence of (a);

wherein said B-cell epitope-containing peptide is not the complete tetanus toxin beta chain;

(ii) said T-cell epitope-containing antigen comprises a CD8+ T cell cancer epitope and a CD4+ T cell cancer epitope, wherein the CD8+ T cell cancer epitope is selected from any one of SEQ ID NOs: 2-6, or an amino acid sequence with at least 65% sequence identity thereto; and the CD4+ T cell cancer epitope is selected from any one of SEQ ID NOs: 7-11, or an amino acid sequence with at least 75% sequence identity thereto; and

(iii) the N-terminus of said T-cell epitope-containing antigen is conjugated to said B-cell epitope-containing peptide.

Thus a Type C conjugate is similar to a Type A conjugate, comprising T cell epitopes which may be utilised in Type A conjugates (as described above), but differs in that the T-cell epitope-containing antigen lacks a translocation peptide. It is preferred that in the T-cell epitope-containing antigen of a Type C conjugate the T cell epitopes are arranged such that the CD8+ T cell epitope is N-terminal to the CD4+ T cell epitope. As detailed above, the T-cell epitope-containing antigen may comprise a protease cleavage site between the T cell epitopes, which may be provided by a spacer. Variation in the T cell epitope sequences (as defined by sequence identity) may also be as described above in respect of Type A conjugates.

Preferably, the CD8+ and CD4+ T cell epitopes are paired in the T-cell epitope-containing antigen of a Type C conjugate as described in respect of the Type A conjugates. Thus in one embodiment a Type C conjugate comprises the CD8+ T cell epitope of SEQ ID NO: 2 or an amino acid sequence with at least 65% sequence thereto, and the CD4+ T cell epitope of SEQ ID NO: 7 or an amino acid sequence with at least 75% sequence identity thereto. An exemplary peptide comprising these epitopes is set forth in SEQ ID NO: 106. Thus in this embodiment the Type C conjugate may comprise a T-cell epitope-containing antigen comprising the amino acid sequence set forth in SEQ ID NO: 106, or an amino acid sequence with at least 70, 75, 80, 85, 90 or 95% sequence identity thereto.

In another embodiment a Type C conjugate comprises the CD8+ T cell epitope of SEQ ID NO: 3 or an amino acid sequence with at least 65% sequence thereto, and the CD4+ T cell epitope of SEQ ID NO: 8 or an amino acid sequence with at least 75% sequence identity thereto. An exemplary peptide comprising these epitopes is set forth in SEQ ID NO: 107. Thus in this embodiment the Type C conjugate may comprise a T-cell epitope-containing antigen comprising the amino acid sequence set forth in SEQ ID NO: 107, or an amino acid sequence with at least 70, 75, 80, 85, 90 or 95% sequence identity thereto.

In another embodiment a Type C conjugate comprises the CD8+ T cell epitope of SEQ ID NO: 4 or an amino acid sequence with at least 65% sequence thereto, and the CD4+ T cell epitope of SEQ ID NO: 9 or an amino acid sequence with at least 75% sequence identity thereto. An exemplary peptide comprising these epitopes is set forth in SEQ ID NO: 108. Thus in this embodiment the Type C conjugate may comprise a T-cell epitope-containing antigen comprising the amino acid sequence set forth in SEQ ID NO: 108, or an amino acid sequence with at least 70, 75, 80, 85, 90 or 95% sequence identity thereto.

In another embodiment a Type C conjugate comprises the CD8+ T cell epitope of SEQ ID NO: 5 or an amino acid sequence with at least 65% sequence thereto, and the CD4+ T cell epitope of SEQ ID NO: 10 or an amino acid sequence with at least 75% sequence identity thereto. An exemplary peptide comprising these epitopes is set forth in SEQ ID NO: 109. Thus in this embodiment the Type C conjugate may comprise a T-cell epitope-containing antigen comprising the amino acid sequence set forth in SEQ ID NO: 109, or an amino acid sequence with at least 70, 75, 80, 85, 90 or 95% sequence identity thereto.

In another embodiment a Type C conjugate comprises the CD8+ T cell epitope of SEQ ID NO: 6 or an amino acid sequence with at least 65% sequence thereto, and the CD4+ T cell epitope of SEQ ID NO: 11 or an amino acid sequence with at least 75% sequence identity thereto. An exemplary peptide comprising these epitopes is set forth in SEQ ID NO: 110. Thus in this embodiment the Type C conjugate may comprise a T-cell epitope-containing antigen comprising the amino acid sequence set forth in SEQ ID NO: 110, or an amino acid sequence with at least 70, 75, 80, 85, 90 or 95% sequence identity thereto.

All other aspects of the Type C conjugates (e.g. the B-cell epitope-containing peptide, conjugation etc.) may be as described above for the Type A conjugates.

The T-cell epitope-containing antigens of the conjugates of the invention may be synthesised by any method known in the art, as detailed above with respect to the B-cell epitope-containing peptides. The T-cell epitope-containing antigens may be chemically synthesised in a non-biological system. Liquid-phase synthesis or solid-phase synthesis, such as Boc or Fmoc synthesis, may be used to generate a desired T-cell epitope-containing antigen.

As described above, the B-cell epitope-containing peptides and T-cell epitope-containing antigens in the conjugates of the invention are defined by sequence identity. Sequence identity may be assessed by any conventional method. The degree of sequence identity between sequences may be determined by computer programmes that make pairwise or multiple alignments of sequences. For instance EMBOSS Needle or EMBOSS stretcher (both Rice, P. et al. (2000), Trends Genet. 16, (6) pp 276-277) may be used for pairwise sequence alignments while Clustal Omega (Sievers F et al. (2011), Mol. Syst. Biol. 7:539) or MUSCLE (Edgar, R. C. (2004), Nucleic Acids Res. 32(5):1792-1797) may be used for multiple sequence alignments, though any other appropriate programme may be used. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

For the purposes of this application, where there is dispute between sequence identity values obtained by different methods, the value obtained by global pairwise alignment using EMBOSS Needle with default parameters shall be considered valid.

In embodiments of the invention which include amino acid sequences which have less than 100% sequence identity to the reference sequences provided (i.e. variant sequence), the modification of the reference sequence to yield the variant sequence may be achieved by addition, deletion or substitution of one or more amino acid residues.

When a sequence is modified by substitution of a particular amino acid residue, the substitution may be a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). A conservative amino acid substitution may be considered to be a substitution in which a particular amino acid residue is substituted for a different amino acid in the same family. A substitution of an amino acid residue may alternatively be a non-conservative substitution, in which one amino acid is substituted for another with a side-chain belonging to a different family.

Amino acid substitutions or additions within the scope of the invention may be made using a proteinogenic amino acid encoded by the genetic code, a proteinogenic amino acid not encoded by the genetic code, or a non-proteinogenic amino acid. Any amino acid substitution or addition may be made using a proteinogenic amino acid. The amino acids making up the sequence of the peptides disclosed herein may include amino acids which do not occur naturally, but which are modifications of amino acids which occur naturally. Provided that these non-naturally occurring amino acids do not alter the sequence and do not affect function, they may be used to generate the peptides described herein without reducing sequence identity, i.e. are considered to provide an amino acid of the peptide. For example, derivatives of amino acids such as methylated amino acids may be used.

A further aspect of the invention is a vaccine composition comprising at least one conjugate of the invention selected from any one of Conjugate I, Conjugate II, Conjugate III, Conjugate IV, and Conjugate V, optionally in combination with Conjugate VI, together with one or more pharmaceutically-acceptable diluents, carriers or excipients. Thus the vaccine composition may comprise any one of Conjugate I, Conjugate II, Conjugate III, Conjugate IV, or Conjugate V. Alternatively the vaccine composition may comprise two or more of Conjugates I-VI, i.e. 2, 3, 4, 5 or 6 of Conjugates I-VI, in any combination.

One embodiment of the invention is a vaccine composition comprising Conjugate I, Conjugate II, Conjugate III, Conjugate IV and Conjugate V.

Another embodiment of the invention is a vaccine composition comprising Conjugate I, Conjugate II, Conjugate III, Conjugate IV, Conjugate V and Conjugate VI.

A further embodiment of the invention is a vaccine composition comprising Conjugate I, Conjugate II, Conjugate IV and Conjugate V.

Yet a further embodiment of the invention is a vaccine composition comprising Conjugate I, Conjugate III, and Conjugate V.

A further embodiment of the invention is a vaccine composition comprising Conjugate I, Conjugate III, Conjugate IV and Conjugate V.

Yet a further embodiment of the invention is a vaccine composition comprising one single conjugate selected based on the genetic profile of the patient and the tumour.

A single type of each conjugate according to the invention may be present in the vaccine composition (i.e. each Conjugate I is identical, each Conjugate II is identical, each Conjugate III is identical, each Conjugate IV is identical, each Conjugate V is identical and each Conjugate VI is identical). Alternatively, multiple types of at least one of the conjugates of the invention may be present in the vaccine composition (i.e. at least 2 different conjugates of at least one of Conjugates I-VI may be present). In some embodiments multiple types of each conjugate of the invention are present (i.e. at least 2 different conjugates of each of Conjugates I-VI are present).

The CD8+ T-cell cancer epitope of SEQ ID NO: 2 is recognised by HLA-A24, those of SEQ ID NOs: 3-4 are recognised by HLA-A2, that of SEQ ID NO: 5 is recognised by HLA-A1 and that of SEQ ID NO: 6 is recognised by HLA-A3, HLA-A11, HLA-A31 and HLA-A33. In central Europe, the frequencies of these HLA alleles in the population are as follows:

| HLA-A Allele | Frequency |
|---|---|
| HLA-A1 | 28% |
| HLA-A2 | 50% |
| HLA-A3 | 29% |
| HLA-A11 | 10% |
| HLA-A24 | 18% |
| HLA-A31 | 5% |
| HLA-A33 | 2% |
| At least one of the above 7 | 96% |

By “frequency” is meant the proportion of individuals in the population who carry each allele. In the analysed population, the vast majority of people carry at least one of the HLA-A alleles bound by the CD8+ T-cell epitopes carried by Conjugates I-V. By identifying multiple CD8+ T-cell epitopes which, combined, recognise the most common HLA-A alleles, an efficacious vaccine may be generated or can be selected for a given individual.

A vaccine composition of the invention may be formulated in any conventional manner according to techniques and procedures known in the pharmaceutical art. “Pharmaceutically acceptable” as used herein refers to ingredients that are compatible with other ingredients of a vaccine composition of the invention as well as physiologically acceptable to the recipient. The nature of the composition and carriers or excipient materials etc. may be selected in routine manner according to choice and the desired route of administration, purpose of treatment etc.

Liquid vaccine compositions, whether they be solutions, suspensions or other like form, may include one or more of the following: sterile diluents such as water for injection, saline solution, preferably physiological, Ringer's solution, isotonic sodium chloride, fixed oils such as synthetic mono- or diglycerides which may serve as a solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as sodium chloride or dextrose. A parenteral preparation can be enclosed in ampoules, disposable syringes or multiple dose vials made of glass or plastic. An injectable pharmaceutical composition is preferably sterile.

The vaccine composition may also comprise one or more adjuvants. Common adjuvants which may be comprised within the vaccine composition include aluminium salts, such as aluminium phosphate and aluminium hydroxide, QS-21 and squalene.

Other commonly used vaccine components are known in the art and include e.g. α-tocopherol and human serum albumin. One or more buffers may also be used to regulate the pH of the composition, e.g. sodium or potassium phosphate, disodium adipate, succinic acid, sodium hydroxide/hydrochloric acid, histidine, sodium borate or trometamol.

The invention further provides a conjugate or vaccine composition of the invention for use in therapy. By “therapy” as used herein is meant the treatment of any medical condition in a subject. Such treatment may be prophylactic (i.e. preventative), or therapeutic, including curative (or intended to be curative), or palliative (i.e. treatment designed merely to limit, relieve or improve the symptoms of a condition). Therapeutic treatment includes any medical treatment of a medical condition, that is a treatment which gives, or intends to give any clinical benefit to a subject having the condition. A subject, as defined herein, refers to any mammal, e.g. a farm animal such as a cow, horse, sheep, pig or goat, a pet animal such as a rabbit, cat or dog, or a primate such as a monkey, chimpanzee, gorilla or human. Most preferably the subject is a human. In particular, the subject may be a male human (a man).

One aspect of the invention is a conjugate or a vaccine composition as herein described and claimed, for use in the prevention or treatment of cancer. Yet another aspect of the invention is a method for the prevention or treatment of cancer in a subject in need of such prevention or treatment, comprising administering to said subject a therapeutically effective amount of a conjugate of the invention, or a vaccine composition comprising a conjugate as herein described and claimed. Yet another aspect of the invention is the use of a conjugate or a vaccine composition as herein described and claimed in the manufacture of a medicament for use in the prevention or treatment of cancer.

Cancer is defined broadly herein to include any neoplastic condition, whether malignant, pre-malignant or non-malignant. Both solid and non-solid tumours are included. The term “cancer cell” is synonymous with “tumour cell”.

As used herein, the cancer may be any cancer in which any epitope carried by one or more of the conjugates of the invention is produced or up-regulated (or more specifically in which any protein containing an epitope carried by one or more of the conjugates of the invention, or an epitope from which an epitope carried by one or more of the conjugates of the invention is derived, is up-regulated). Cancers which may be treated by the methods of the invention include melanoma, multiple myeloma, gastric cancer, ovarian cancer, prostate cancer, testicular cancer, breast cancer, bladder or urothelial cancer, oesophageal cancer, oral cancer and lung cancer. By prostate cancer is meant both primary prostate cancer (i.e. prostate cancer which is localised to the prostate) and metastatic prostate cancer. In one aspect of the invention, a conjugate or a vaccine composition as described and claimed herein may be used to treat metastases of prostate cancer located elsewhere (i.e. not in the prostate) in the body of the subject.

Conjugates or vaccines of the invention may also be useful in the treatment of localised prostate cancer, i.e. prostate cancer that has not yet spread or metastasised to other areas of the body. In one embodiment of the invention, a conjugate or a vaccine composition as described and claimed herein may be useful in the treatment of localised prostate cancer in subjects who are at risk (e.g. intermediate/high risk) of metastasis or of subjects at risk of relapse of prostate cancer, for example to delay or prevent relapse. A further embodiment of the invention is the use of a conjugate or a vaccine composition as herein disclosed and claimed in immunotherapy for cancer.

Importantly, the subject to which the conjugate(s) or vaccine composition of the invention is to be administered preferably has pre-existing antibodies against TTx, and more specifically to SEQ ID NO: 1. Whether an intended subject has antibodies against TTx or SEQ ID NO: 1 can be determined by e.g. a Tettox ELISA, described above. If the intended subject does not have anti-TTx antibodies, the subject can receive a vaccination against tetanus comprising TTd, to drive anti-TTx antibody production in the subject. The methods of treatment provided in the invention thus include the administration of a vaccine to induce an immune response against TTx prior to administering the conjugate(s) or vaccine composition of the invention. The vaccine to induce an immune response to TTx may be administered at least 1, 2, 3, 4, 5, 6, 7, 8, 9, 10, 20, 30 or more weeks prior to administering the conjugate(s) or vaccine composition of the invention, and may comprise TTd.

Alternatively, if an intended subject does not have anti-TTx antibodies, the conjugate(s) or vaccine composition can be administered in combination with exogenous anti-TTx antibodies, to provide the subject with a passive humoral immune response to TTx. For instance, the conjugate(s) or vaccine composition of the invention can be administered to a subject in combination with (i.e. at the same time as, or shortly before or after) a solution or serum comprising anti-TTx antibodies, e.g. Tetaquin or any equivalent anti-TTx antibody preparation.

A conjugate or a vaccine composition as herein described and claimed, may be administered to a subject by a parenteral route, e.g. the administration may be subcutaneous, intramuscular, intravenous, intraarterial, intraperitoneal, intralesional or intradermal administration. Administration as a bolus injection may be useful.

By the term “therapeutically effective amount” is meant an amount of the therapeutically active agent which is sufficient to show benefit to the condition of the subject, such as slowing down or inhibiting the growth of the cancer, or even cause the cancer to reduce in size.

The methods of treatment of the invention may further comprise the administration of a second or further therapeutically active agent, such as an anti-cancer agent. The second or further therapeutically active agent may for instance be a chemotherapeutic agent or a further immunotherapeutic agent, e.g. an antibody or re-directed T-cell which targets a cancer antigen. Alternatively the second or further therapeutically active agent may be e.g. an antibiotic, an antiviral or antifungal agent, or an immuno-modulatory agent as discussed above. Alternatively or additionally the methods of treatment of the invention may be combined with other therapies such as surgery, hormone therapy and/or radiotherapy.

A further aspect of the invention is a kit comprising a conjugate or a vaccine composition as herein disclosed and claimed, in combination with a second therapeutically active agent, e.g. an agent as defined above. When the kit comprises both a conjugate or vaccine composition of the invention and a second therapeutically active agent, the conjugate/composition and the second agent may be for separate, sequential or simultaneous administration to a subject. Such a kit may alternatively be defined as a combination or a combined product. The kit may be for use in therapy, in particular for use in cancer therapy.

Thus in a further aspect, the invention also provides a conjugate or a vaccine composition as defined herein and a second therapeutically active agent (more particularly a second anti-cancer agent) as a combined preparation for separate, sequential or simultaneous use in therapy, such as in the treatment or prevention of cancer.

The invention also provides a polypeptide comprising or consisting of an amino acid sequence set forth in any one of SEQ ID NOs: 13-17, or an amino acid sequence with at least 70, 75, 80, 85, 90 or 95% sequence identity thereto, wherein said polypeptide comprises from N-terminus to C-terminus:
(a) a translocation peptide;
(b) a CD8+ T-cell cancer epitope; and
(c) a CD4+ T-cell cancer epitope;
   wherein a proteasome cleavage site is optionally present between said CD8+ T-cell cancer epitope and said CD4+ T-cell cancer epitope, optionally wherein said cleavage site is provided by a spacer;

wherein said translocation peptide is able to mediate TAP-driven transport of said polypeptide or said CD8+ T-cell cancer epitope into the endoplasmic reticulum of a host cell.

As can be seen, this group of polypeptides of the invention correspond exactly to the T-cell epitope-containing antigens of Conjugates I-V (when said T-cell epitope-containing antigens comprise or consist of an amino acid sequence set forth in any one of SEQ ID NOs: 13-17, or an amino acid sequence with at least 70% sequence identity thereto, respectively), and thus all discussion of the T-cell epitope-containing antigens of those conjugates apply equally to these polypeptides of the invention.

In another aspect the invention provides a polypeptide comprising or consisting of an amino acid sequence set forth in any one of SEQ ID NOs: 106-110, or an amino acid sequence with at least 70, 75, 80, 85, 90 or 95% sequence identity thereto, wherein said polypeptide comprises from N-terminus to C-terminus:
(a) a CD8+ T-cell cancer epitope; and
(b) a CD4+ T-cell cancer epitope;

wherein an optional proteasome cleavage site can be present between said CD8+ T-cell cancer epitope and said CD4+ T-cell cancer epitope.

As can be seen, this group of polypeptides of the invention correspond exactly to the T-cell epitope-containing antigens of the Type C conjugates of the invention described above), and thus all discussion of the T-cell epitope-containing antigens of those conjugates apply equally to these polypeptides of the invention.

As defined herein, a polypeptide (e.g. a polypeptide of the invention) comprises amino acids joined by peptide bonds. A polypeptide, as defined herein, may also comprise one or more non-peptidic moieties. That is to say, a “polypeptide” as defined herein may consist of amino acids joined by peptide bonds, but alternatively may additionally comprise non-amino acid and/or non-peptide moieties. Any chemical moiety may be included in a polypeptide as defined herein, including for instance a carrier or functional group. Thus, a polypeptide of the invention may include also a polypeptidic compound. In a particular embodiment the polypeptidic compound comprises a polypeptide of the invention joined to a carboxylic acid azide, such as a hexanoyl azide moiety (i.e. the polypeptidic compound of the invention may have a structure as shown above in Formula IV). Other useful carboxylic acid azides include azidopropionic acid and the like.

The invention further provides a nucleic acid molecule comprising or consisting of a nucleotide sequence encoding a polypeptide of the invention. The genetic code is well-known so the skilled person will easily be able to generate a nucleic acid molecule of the invention based on the encoded polypeptide sequences provided. The nucleic acid molecule of the invention may be an isolated nucleic acid molecule and may include DNA (including cDNA) or RNA or chemical derivatives of DNA or RNA, including molecules having a radioactive isotope or a chemical adduct such as a fluorophore, chromophore or biotin (“label”). Thus the nucleic acid may comprise modified nucleotides. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phosphoraniladate and phosphoroamidate. The term “nucleic acid molecule” specifically includes single- and double-stranded forms of DNA and RNA.

Such a molecule may be generated by recombinant means or by chemical synthesis, e.g. solid-phase synthesis using the phosphoramidite method.

The invention further provides a construct, e.g. a recombinant construct, comprising a nucleic acid molecule of the invention. The nucleic acid molecule may be operably linked within said construct to an expression control sequence. Such an expression control sequence will typically be a promoter. Accordingly, the construct may comprise a promoter. Optionally, the construct may additionally contain a further one or more polypeptide-coding sequences and/or one or more regulatory sequences. The optional one or more polypeptide-coding sequences may be under the control of the same promoter or under the control of a different promoter. It is therefore encompassed in the present invention for a construct to encode more than one polypeptide of the invention. In this aspect, the construct may comprise two or more nucleic acid sequences of the invention.

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e. the coding sequence is under the transcriptional control of the promoter). Coding sequences may be operably linked to regulatory sequences in sense or antisense orientation.

The term “regulatory sequences” refers to nucleotide sequences located upstream (5′ non-coding sequences), within, or downstream (3′ non-coding sequences) of a coding sequence, and which influence transcription, RNA processing or stability, or translation of the associated coding sequence. Regulatory sequences may include promoters, operators, enhancers and translation leader sequences. As used herein, the term “promoter” refers to a nucleotide sequence capable of controlling the expression of a coding sequence or RNA. In general, a coding sequence is located 3′ to a promoter sequence. Promoters may be derived in their entirety from a native gene, or be composed of different elements derived from different promoters found in nature, or even comprise synthetic nucleotide segments. It is further recognised that since in most cases the exact boundaries of regulatory sequences have not been completely defined, nucleic acid fragments of different lengths may have identical promoter activity.

In another aspect the invention provides a vector comprising a nucleic acid molecule or construct of the invention. Vectors comprising one or more of the nucleic acid molecules (or constructs) of the invention may be constructed. The choice of vector may be dependent on the host organism or cell(s) in which the nucleic acid molecule of the invention is to be expressed, the method that will be used to transform the host cell(s), and/or the method that is to be used for protein expression (or any another intended use of the vector). The skilled person is well aware of the genetic elements that must be present in a vector in order to successfully transform, select and propagate cells containing a nucleic acid or construct of the invention. The skilled person will also recognise that different independent transformation events will result in different levels and patterns of expression and thus that multiple events may need to be screened in order to obtain cells displaying the desired level of expression. Such screening may be accomplished by Southern analysis of DNA, Northern analysis of mRNA, Western analysis of protein etc.

Also included in the scope of the invention are methods for the production of a conjugate of the invention, and in particular methods for the production of such conjugates which contain one or more, or more particularly two or more, B-cell epitope-containing peptides, and in which the B-cell epitope-containing peptide and T-cell epitope-containing antigen are conjugated by each being coupled, or linked, to a core compound as a linker moiety.

Accordingly, in another aspect the invention provides a method of producing a conjugate of the invention, as set forth above.

One embodiment of the invention is a method for making a conjugate of the invention, comprising the steps of:

(i) providing a core compound being a tri-amino-2,2-dimethyl propanoic acid linker compound comprising a diphenylcyclooctyne PEG spacer wherein the three amino groups are functionalised with propionyl maleimide groups;

(ii) providing three B-cell epitope-containing peptides as defined herein, wherein the peptide molecules comprise a thiol group at the C-terminus, preferably wherein said thiol group is the side-chain of a cysteine residue at the C-terminus of the B-cell epitope-containing peptide;

(iii) attaching the three B-cell epitope-containing peptides to the core compound of step (i) to generate an adduct, by forming a succinimidyl thioether between each maleimide ring of the core compound and a thiol group of a peptide molecule;

(iv) providing a T-cell epitope-containing antigen, wherein the antigen comprises a N-terminal azido carboxylic acid group;

(v) attaching the azido carboxy-antigen of (iv) to the adduct resulting from step (iii); and

(vi) opening the succinimide rings of the adduct, wherein said ring opening may occur before or after step (iii).

In yet another embodiment, the invention provides a method of producing a conjugate of the invention, comprising:

(i) synthesising an intermediate compound comprising tri-amino-2,2-dimethyl propanoic acid with a diphenylcyclooctyne PEG spacer, optionally wherein each of the three amino groups of the tri-amino-2,2-dimethyl propanoic acid is mono-substituted with a protecting group, preferably wherein said protecting group is a Boc group;

(ii) when said amino groups of the intermediate compound are mono-substituted with a protecting group, deprotecting the amino groups;

(iii) reacting the intermediate compound of step (i) or (ii) with maleimide propanoic acid-O-succinimide ester to attach a maleimide ring to each unprotected amino group, thereby to form a core compound;

(iv) conjugating three B-cell epitope-containing peptides as defined herein to the core compound of step (iii), by formation of a succinimidyl thioether between each maleimide ring of the core compound and a thiol group of a B-cell epitope-containing peptide, preferably wherein said thiol group is the side-chain of a cysteine residue of the B-cell epitope-containing peptide;

(v) coupling a T-cell epitope-containing antigen as defined herein to an azido carboxylic acid;

(vi) conjugating the azido carboxy-antigen of step (v) to the compound of step (iv); and

(vii) opening the succinimide rings of the central core, wherein said ring opening may occur before or after step (vi).

The intermediate compound produced in step (i) may be synthesised as demonstrated in the Examples below. The intermediate compound produced in step (i) may have the structure shown in Formula I above; alternatively, if the amino groups are protected with Boc (tert-butyloxycarbonyl) it has the structure shown in Formula VIII, below:

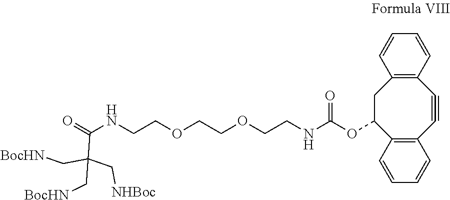

The B-cell epitope-containing peptide used in the conjugation may be any such peptide defined herein. In particular it may comprise an amino acid sequence set forth in any one of SEQ ID NOs: 1 and 30-86 or an amino acid sequence with at least 70, 75, 80, 85, 90, 95, 96, 97, 98 or 99% sequence identity thereto. The B-cell epitope-containing peptide may comprise the amino acid sequence set forth in SEQ ID NO: 21 or an amino acid sequence with at least 70, 75, 80, 85, 90, 95, 96, 97, 98 or 99% sequence identity thereto. The thiol group of the B-cell epitope-containing peptide used to conjugate the peptide to the central core may be the thiol side chain of a cysteine residue which forms the C-terminus of the B-cell epitope-containing peptide.

The T-cell epitope-containing antigen used in the synthesis may be any T-cell epitope-containing antigen as defined herein, in particular it may comprise an amino acid sequence set forth in any one of SEQ ID NOs: 13-17 or 19-20, or an amino acid sequence with at least 70, 75, 80, 85, 90, 95, 96, 97, 98 or 99% sequence identity thereto. The T-cell epitope-containing antigen may be conjugated in step (v) to any azido carboxylic acid, e.g. azido hexanoic acid, azido pentanoic acid, azido butanoic acid or azido propanoic acid. The azido carboxy-antigen is conjugated to the core compound at the site of the carbon-carbon triple bond. The opening of the succinimide rings of the core compound occurs after conjugation of the B-cell epitope-containing peptides to the maleimide rings (thus yielding the succinimide rings), but may occur either before or after the azide-containing moiety comprising the T-cell epitope-containing antigen is conjugated to the core compound.

The present invention may be more fully understood from the non-limiting Examples below and in reference to the drawings, in which:

FIG. 1 shows exemplary reaction schemes for synthesising conjugates of the invention, following the protocols described in Example 2. As shown in the reaction scheme, compound 12 may be either conjugated directly to an SLP, yielding a closed-ringed conjugate of the invention (Conjugate 14) or may first undergo ring-opening and then be conjugated to an SLP, to yield an open-ringed conjugate exemplified by Conjugate 16. Thus the two reaction pathways shown from compound 12 are alternative pathways, one of which yields open-ringed conjugates and the other closed-ringed conjugates.

FIG. 2 shows cytokine (TNFα and IFNγ) production by T-cells in donor blood, as analysed by flow cytometry. Peptides and conjugates were incubated in human whole blood from prostate cancer patients and healthy donors, pre- and post DTP vaccination (A) or with and without a mouse anti-MTTE IgG2a antibody (B). The change for each individual donor is shown, the result pre-vaccination (or without anti-MTTE antibody, left) being linked to the result post-vaccination (or with anti-MTTE antibody, right). The cells were gated as CD45RO+CD3+CD4+CD8− or CD45RO+CD3+CD4−CD8+, and the % of IFNγ+ and TNFα+ cells are displayed. The blood was either untreated (0 time point) or treated with saline solution (NaCl), Conjugates I-VI of the invention (LUR1-6) or the T-cell epitope-containing antigens of Conjugates I-VI (SLP1-6). The [MTTE]₃-NLV conjugate containing the HLA-A*0201-restricted epitope pp65(NLV) from CMV and CMV lysate were used as positive controls, and the MTTE3-irrelevant (MTTE3-irrel) conjugate was used as a further control. This conjugate contained a scrambled SLP sequence (DGLQGLLLGLRQRIETLEGK, SEQ ID NO: 88) without any known human T cell epitopes. The dot plots of the three responding donors from A and B are displayed in C and D.

FIG. 3 shows the titres of anti-MTTE antibodies in cancer patients' plasma before and after receipt of a DTP booster. FIG. 3A shows the titre of total IgG antibodies, FIG. 3B the titre of IgM antibodies, FIG. 3C the titre of IgG1 antibodies and FIG. 3D the titre of IgG4 antibodies. **equals p-value <0.01 and ns=non-significant, assessed by paired t-test.

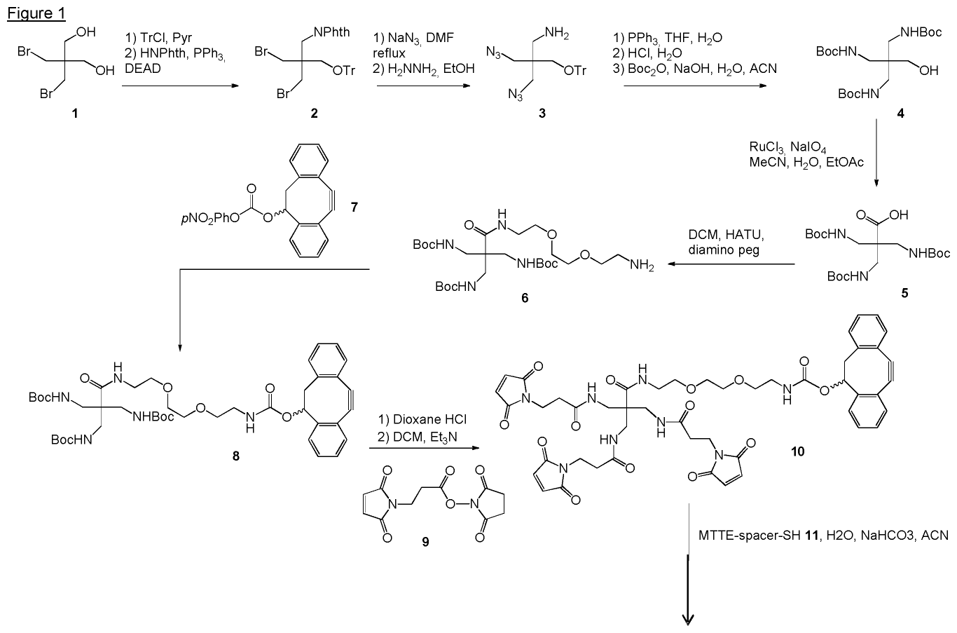

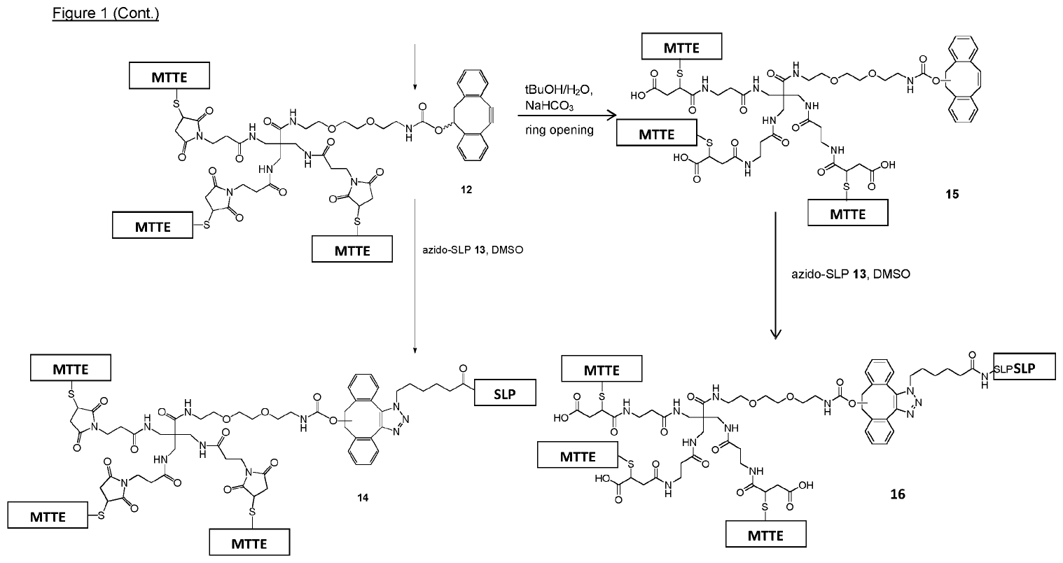

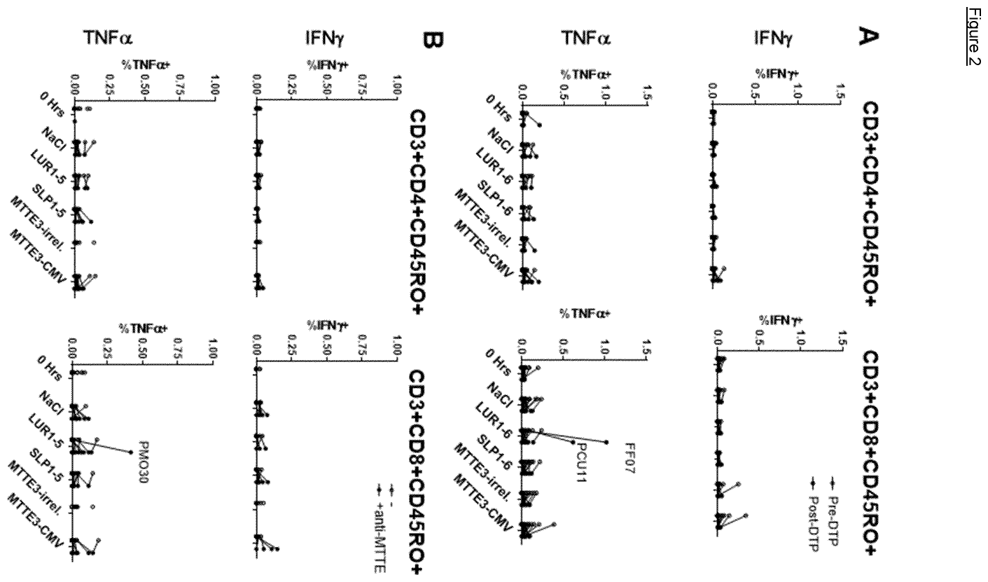

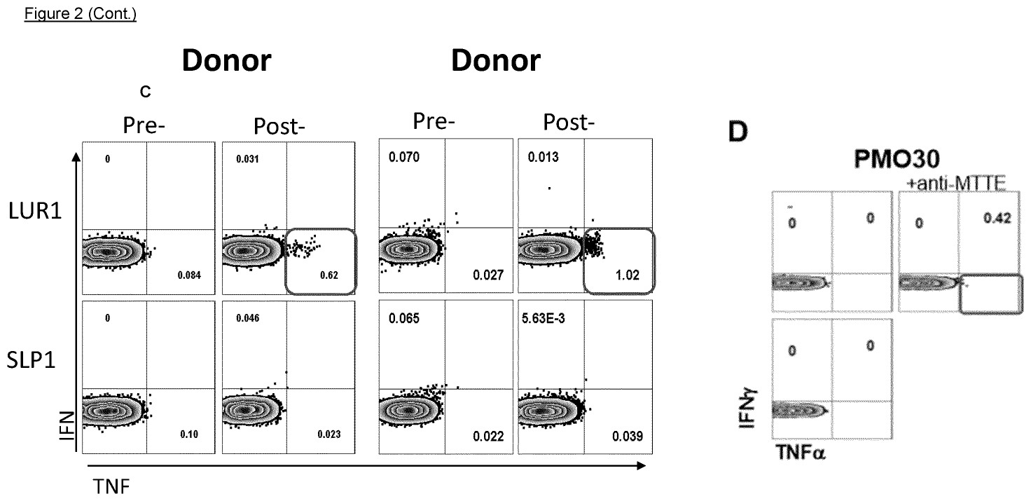

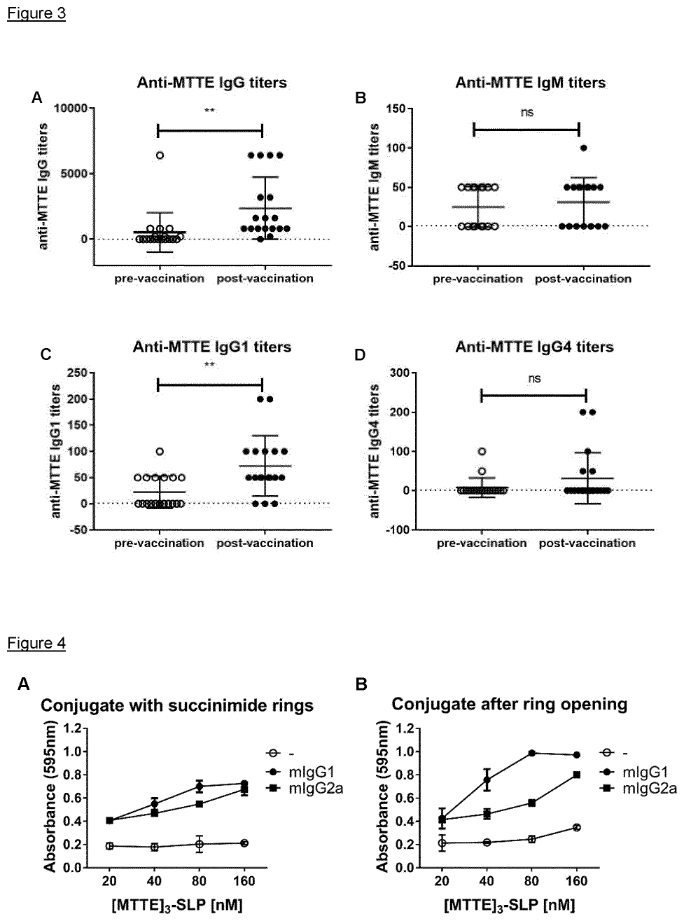

FIG. 4 shows the results of in vitro antigen presentation experiments, using antigen provided in constructs synthesised according to Examples 2 and 3. FIG. 4A shows T-cell activation levels using various concentrations of a conjugate with intact succinimide rings; FIG. 4B shows T-cell activation levels using various concentrations of an equivalent conjugate in which the succinimide rings have been opened.

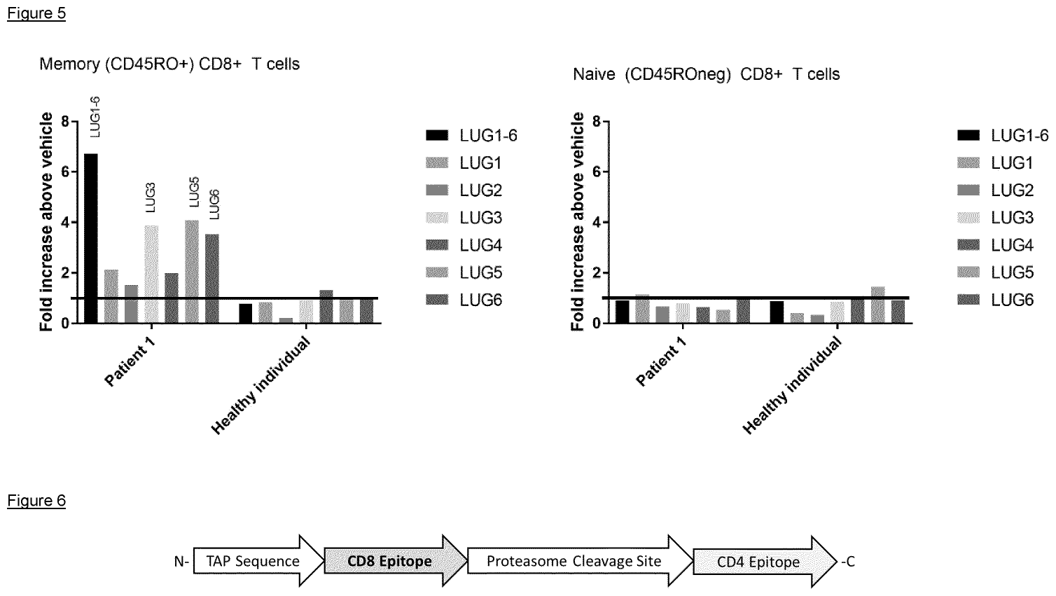

FIG. 5 shows cytokine (IFNγ) production by memory (CD45RO+) or non-memory (CD45RO−) CD8+ T-cells in donor blood from a patient that responded in FIG. 2. The blood is either untreated (0 time point) or treated with saline solution (NaCl). Blood from the donor was subjected to either a mix of conjugates I-VI of the invention (LUR1-6) or each individual conjugate was assessed alone. Results are shown as the fold increase of IFNγ production compared to vehicle-exposed blood.

FIG. 6 is a schematic diagram showing the general structure of SLP1-6, the T-cell epitope-containing antigens of Conjugates I-VI respectively. Each SLP contains the same TAP sequence, but the CD8 and CD4 epitopes, and the proteasome cleavage site, differ between the SLPs. The N- and C-termini of the SLPs are indicated.

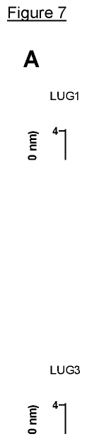

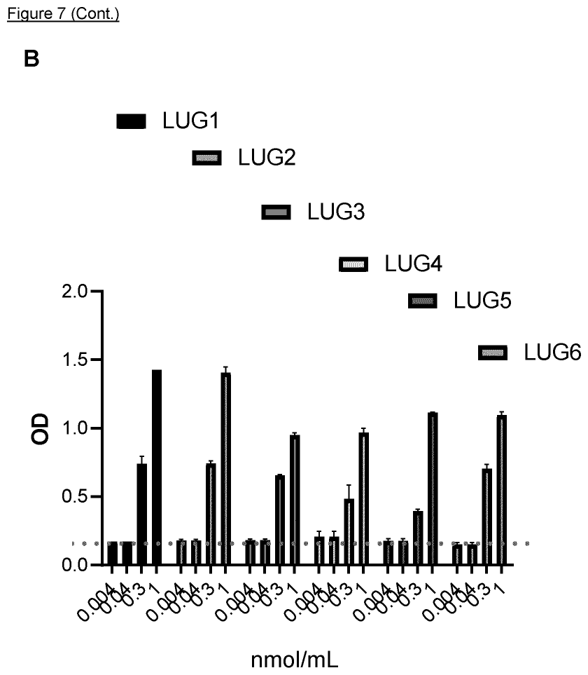

FIG. 7 shows the binding of GMP LUG1-6 constructs to human anti-MTTE antibodies. A shows binding of conjugates to monoclonal human IgG1 anti-MTTE antibodies. Conjugates were coated onto an ELISA plate at a range of concentrations (from 0.000457-1 nmol/ml). Human recombinant anti-MTTE IgG1 antibody was used as primary antibody and detection was performed using an anti-human kappa light chain secondary antibody. B shows binding of conjugates to polyclonal human anti-MTTE antibodies from a donor. Conjugates were coated onto an ELISA plate at a range of concentrations (from 0.004-1 nmol/ml) and incubated with diluted donor plasma. Detection was performed using an anti-human kappa light chain secondary antibody.

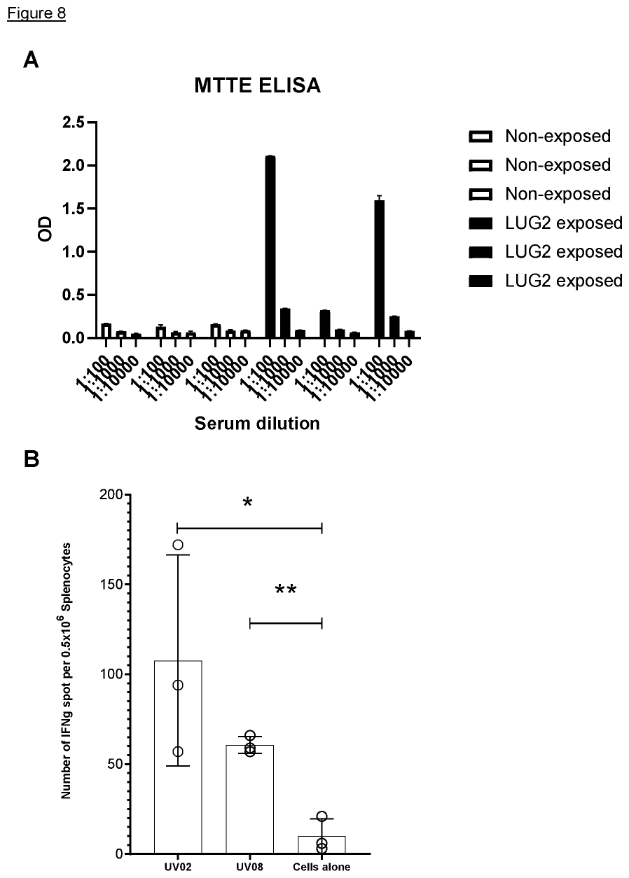

FIG. 8 shows the effects on anti-MTTE titre of vaccination of animals with LUG2 conjugates (A), and (B) ELISPOT analysis of T cell responses after HLA-DR4 mice had been vaccinated with LUG2. HLA-DR4 transgenic mice were subcutaneously vaccinated with LUG2 (20 μg) using a prime/boost schedule. A week later the mice were sacrificed, heart bleed was performed, serum was analysed by anti-MTTE ELISE and the splenocytes were analysed using IFNγ ELISPOT. ELISPOT was performed by incubation of the splenocytes with the SLPs UV02 (SEQ ID NO: 14) and UV08 (SEQ ID NO: 107) for 48 h. SEB was used as positive control and untreated splenocytes as negative control.

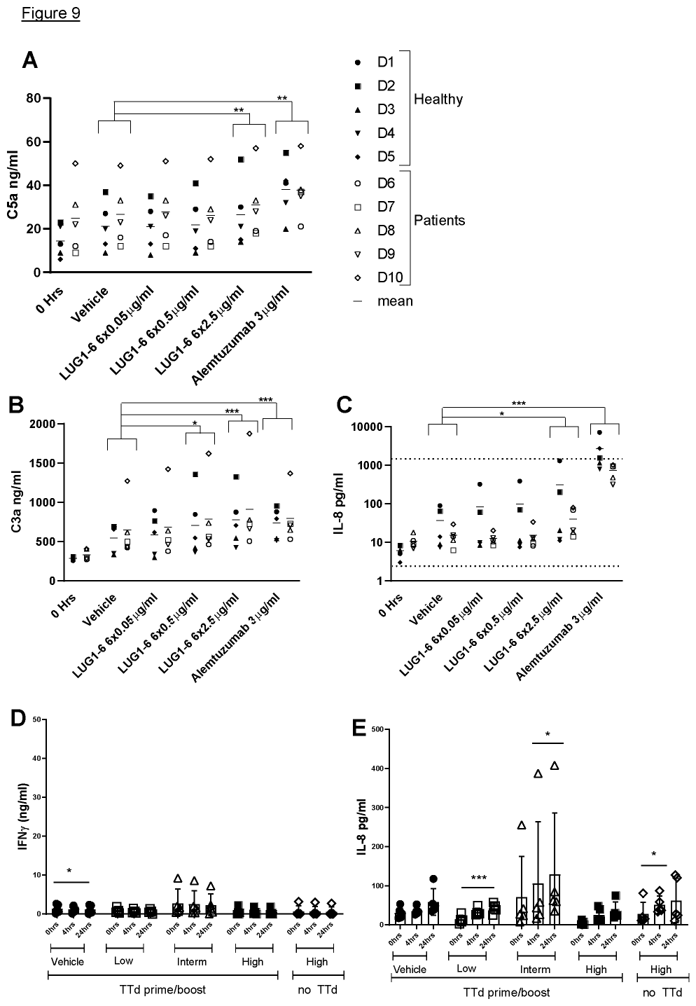

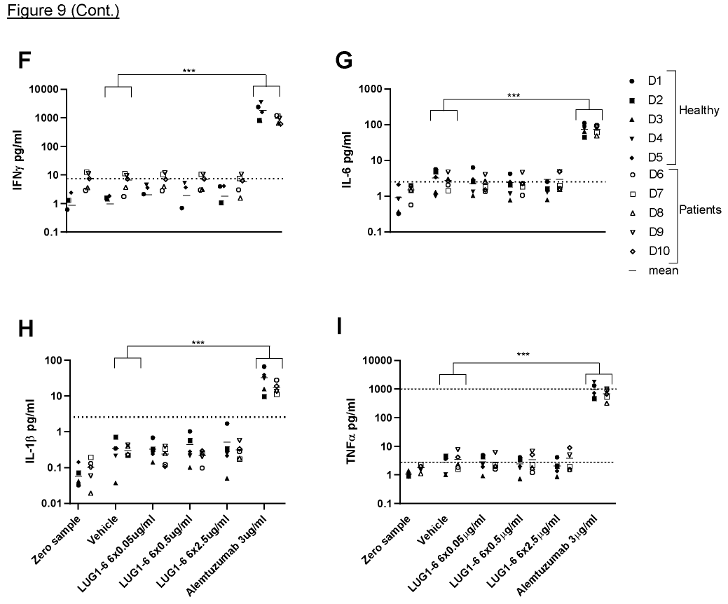

FIG. 9 shows an analysis of TENDU toxicity in a human blood loop assay and in male rabbits (the TENDU vaccine comprises the LUG1-6 conjugates, which as described below correspond to Conjugates I-VI described herein manufactured to GMP standard). Fresh blood from five Boostrix vaccinated prostate cancer patients and five healthy individuals was transferred to the loops. The LUG1-6 constructs were added at the respective concentrations or NaCl was added as a vehicle. Alemtuzumab (3 μg/ml) was added to the respective loops as positive control. Plasma samples were collected at 0 min and 15 min for measurement of C3a and C5a by ELISA (A-B). For analysis of IL-8 (C), IFNγ (F), IL-6 (G), IL-1β (H) and TNFα (I) plasma samples were collected at 0 min and 4 h. Plasma samples were analysed using the MSD array and MSD software. The LLOD and ULOD were defined as described in the methods. Male rabbits were subcutaneously vaccinated four times with Equip-T followed by four subcutaneous TENDU vaccination at either low, intermediate or high dose (See Table VI). The vaccinations were given every two weeks and plasma samples were collected on week 8 and week 15 before the first and last administration of TENDU and 4 h and 24 h after TENDU administration. Plasma was analysed using rabbit ELISA Kits. Concentrations of IFNγ (D) and IL-8 (E) were calculated.

### EXAMPLES

### Example 1—B-Cell Epitope-Containing Peptide Design

A variety of designs of B-cell epitope-containing peptides comprising the amino acid sequence set forth in SEQ ID NO: 1 were synthesised, and analysed to determine a design to allow for antibody binding to the MTTE sequence. As seen previously in the published patent application WO 2011/115483, N-terminal modification hampered antibody binding to the MTTE.

The synthesised peptides were conjugated to biotin (at either their C- or N-terminus). Certain of the peptides included an additional amino acid sequence which formed a spacer between the biotin and the MTTE of SEQ ID NO: 1. The peptides designed are set forth in Table 1, below. A control peptide was also synthesised, comprising a scrambled MTTE sequence with a C-terminal spacer sequence (SEQ ID NO: 103) conjugated to biotin.

TABLE 1

| | | SEQ ID NO |
|---|---|---|
| | | of Amino |
| Biotin | | Acid |
| Location | Peptide Sequence | Sequence |
| C-terminal | F-I-G-I-T-E-L-K-K-L-E-S-K-I-N-K-V-F-S-S-A- | 104 |
| | F-A-D-V-E-A-A | |
| C-terminal | F-I-G-I-T-E-L-K-K-L-E-S-K-I-N-K-V-F | 1 |
| C-terminal | F-I-G-I-T-E-L-K-K-L-E-S-K-I-N-K-V-F-A-A-K- | 105 |
| | Y-A-R-V-R-A | |
| C-terminal | E-K-L-l-N-K-L-S-K-l-F-K-G-T-I-E-V-F-S-S-A- | 103 |
| | F-A-D-V-E-A-A | |
| N-terminal | F-I-G-I-T-E-L-K-K-L-E-S-K-I-N-K-V-F-A-A-K- | 105 |
| | Y-A-R-V-R-A | |

Antibody binding to each peptide was analysed by ELISA. The biotinylated peptides were incubated on a streptavidin-coated Nunc-Immuno MaxiSorp plate. A polyclonal rabbit anti-MTTE antibody batch was used to titrate the titre to each individual peptide coated on the plate. S-shaped curves were calculated using the Bolzmann formula. The titre-value, herein the dilution of 50% of max-absorbance, was extracted from the Bolzmann data of the curve. A goat anti-rabbit IgG conjugated to alkaline phosphatase was used as secondary antibody, and 4-nitrophenyl phosphate disodium salt hexahydrate was used to develop the assay. Absorbance was then read at 405 nm to determine the titre. The results are presented in Table 2, below:

TABLE 2

| Biotin Location | Peptide Sequence | Titre (EC50) |
|---|---|---|
| C-terminal | SEQ ID NO: 104 | 500 |
| C-terminal | SEQ ID NO: 1 | 400 |
| C-terminal | SEQ ID NO: 105 | 800 |
| C-terminal | SEQ ID NO: 103 | 0 |
| N-terminal | SEQ ID NO: 105 | 200 |

The biotinylated peptide without a spacer displays a titre of 400, whereas the two peptides comprising spacers C-terminal to SEQ ID NO: 1 display similar or enhanced titres, indicating that a C-terminal spacer does not negatively influence antibody binding. Conjugation of biotin to the C-terminus of the peptide was found to be important for optimal antibody binding. As shown above, conjugation of biotin to the N-terminus of the MTTE resulted in antibody binding to the MTTE being reduced by half. The scrambled MTTE sequence with a C-terminal spacer does not display any titre, indicating that no antibody bound the peptide, regardless of the inclusion of a spacer.

### Example 2—Conjugate Synthesis

In this Example the synthesis of a construct of the invention is described. This Example relates to the synthesis of a construct containing 3 B-cell epitope-containing peptides, which comprise the MTTE sequence FIGITELKKLESKINKVF (SEQ ID NO: 1) and a C-terminal spacer with the sequence AAKYARVRAKC (SEQ ID NO: 102) (i.e. they have the sequence set forth in SEQ ID NO: 21); and an example T-cell epitope-containing antigen with the sequence LEQLESIINFEKLAAAAAK (SEQ ID NO: 87) derived from ovalbumin (UniProt accession number P01012). The synthesis was performed as described on pp. 40-45 of EP 2547364 B1 (WO 2011/115483). For completeness, the reaction scheme is shown in FIG. 1 (all compound numbers (bold) in this example refer to the compounds of FIG. 1).

Core Synthesis

The core of the conjugate (10) was synthesised as described in [113]-[121] of EP 2547364 B1.

Peptide Synthesis

The two peptides used in this Example were synthesised as described in [122] of EP 2547364 B1. The peptides synthesised were:

(i) F-I-G-I-T-E-L-K-K-L-E-S-K-I-N-K-V-F-A-A-K-Y-
A-R-V-R-A-K-C (MTTE-spacer-SH, peptide 11);
and
(ii) Azidohexanoyl-L-E-Q-L-E-S-I-I-N-F-E-K-L-A-
A-A-A-A-K (azido-antigen, peptide 13):

### Construct Synthesis

Construct 14 was synthesised as described in [123] of EP 2 547 364 E1.

### Ring Opening

Succinimide rings can cause molecular instability, meaning that compounds containing succinimide rings can show limited stability under particular conditions, especially under basic conditions and at elevated temperatures. An assessment of stability of the constructs with succinimide rings (e.g. construct 14) was performed, in which the constructs were incubated at pH 8.7 and 30° C. for 46 hours. Under these conditions, after 46 hours virtually all succinimide rings were hydrolysed, and some of the molecules had lost an MTTE group (data not shown). Thus, to avoid instability issues, an extra incubation step for succinimide ring opening was introduced to the conjugate synthesis pathway, yielding stable constructs.

Open ring constructs were obtained as follows: 10 mg MTTE-spacer-SH (peptide 11) was dissolved in 300 μl Milli-Q water. A solution of core structure 10 in 100 μl acetonitrile was added and the pH adjusted to 6 with 4.2% NaHCO₃. The reaction was allowed to proceed at room temperature for about 1 hour, yielding compound 12. The succinimide rings of compound 12 were then opened as follows: 425 μl tBuOH/water (9:1, v/v) and 100 μl 4.2% NaHCO₃ were added to the reaction mixture containing the newly-synthesised compound 12. The ring opening reaction was allowed to proceed for 72 hours at 30° C. The reaction mixture was brought to pH 6 with 0.5 M acetic acid. Ring opening yields a mixture of 8 open-ringed isomers, as each succinimide ring may be opened such that the sulphide group is adjacent to either the amide bond or the carboxyl group. An example of an open-ringed isomer obtained from ring opening is presented as Compound 15. To this mixture was added azidohexanyl SLP (13) in DMSO, and an open-ringed conjugate comprising an MTTE and T-cell epitope-containing SLP generated (Compound 16, shown, is the conjugate obtained from attachment of an SLP to Compound 15).

### Example 3—Synthesis of Alternative Open-Ringed Conjugates

All construct numbers are the same as in Example 2/FIG. 1.

Constructs 10 and 11 were synthesised as described above. An azido-peptide comprising an antigen with the amino acid sequence:

(SEQ ID NO: 15)
A-R-W-W-S-L-S-L-G-F-L-F-L-A-A-A-G-K-V-F-R-G-N-K-
V-K-N-A-Q-L-A

was synthesised using the same protocol as for the synthesis of compound 13, described above, with the exception that azidopropanoic acid was used instead of azidohexanoic acid.

Compound 12 was synthesised, and its rings opened, as above. To this mixture was added azidopropionyl SLP in DMSO, and open-ringed conjugates comprising an MTTE and T-cell epitope-containing SLP generated. The resultant compounds were analysed by mass spectrometry as described above. The constructs have a calculated mass of 14698.4 Da, and a measured deconvoluted average mass of 14699.0 Da.

### Example 4—Selection of T-Cell Epitope Combinations and TTES

11 known prostate cancer CD8+ T-cell epitopes (C1-11) and 6 known prostate cancer CD4+ T-cell epitopes (H1-H6) were selected from the literature:

| | CTL-epitope | SEQ ID | |
|---|---|---|---|
| Code | sequence | NO: | HLA Allele Bound |
| C1 | ILLWQPIPV | 90 | A2 |
| C2 | YLPFRNCPR | 91 | A3, A11, A31, A33 |
| C3 | LYCESVHNF | 92 | A24 |
| C4 | GMPEGDLVY | 5 | A1 |
| C5 | LLHETDSAV | 3 | A2 |
| C6 | MMNDQLMFL | 93 | A2 |
| C7 | VLAGGFFLL | 94 | A2 |
| C8 | KVFRGNKVK | 6 | A3, A11, A31, A33 |
| C9 | NYARTEDFF | 2 | A24 |
| C10 | LLAVTSIPSV | 95 | A2 |
| C11 | SLSLGFLFL | 4 | A2 |
| | Helper-epitope | SEQ ID | |
| Code | sequence | NO: | HLA Allele Bound |
| H1 | GQDLFGIWSKVYDPL | 7 | Promiscuous |
| H2 | TEDTMTKLRELSELS | 96 | Promiscuous |
| H3 | GKVFRGNKVKNAQLA | 9 | Promiscuous |
| H4 | TGNFSTQKVKMHIHS | 10 | DR4 |
| H5 | NYTLRVDCTPLMYSL | 11 | DR1/DR9 |
| H6 | RQIYVAAFTVQAAAE | 8 | DR4/DR9 |

It was decided that the conjugates of the prostate cancer vaccine would contain a single long peptide (SLP) comprising one each of C1-11 and H1-6, and that the vaccine would comprise 5 such conjugates, each with a different CD8+ and CD4+ epitope. Epitope combinations were selected based on the requirement that a candidate long peptide should contain a CTL-epitope that is properly TAP translocated and of which the C-terminus is generated in the context of the longer peptide also containing the helper T-cell epitope.

SLPs containing all 66 possible combinations of the listed CTL-epitopes and Helper-epitopes were synthesised. These peptides were treated with commercially-available immuno-proteasome according to the protocol of the supplier. Each peptide (1 μl of the DMSO stock solution) was added to 300 μl aqueous buffer containing 0.5 μg immunoproteasome 20S (human, purified, BML-PW9645-0050, Enzo Life Science), 30 mM Tris-HCl (pH 7.2), 10 mM KCl, 5 mM MgCl₂ and 1 mM DTT. The mixture was vortexed and incubated at 37° C. for various time periods. At each time point an aliquot (50 μl) was taken from the digestion mixture, added to 4 μl formic acid and the solution obtained was homogenised by vortexing and stored at −20° C. until analysis. For analysis 1 μl of this solution was mixed with 1 μl matrix solution (10 mg/ml α-cyano-4-hydroxy cinnamic acid (ACH) in acetonitrile/water 1/1 containing 0.2% TFA) and spotted on a MALDI-TOF target plate. Samples at all time points were analysed with MALDI-TOF mass spectrometry (Bruker Microflex) revealing the proteasome-induced peptide fragments (>800 Da).

Proteasomal degradation was monitored after 24 hr digestion using MALDI-TOF mass spectrometry with a Bruker Microflex or a Bruker Ultraflex instrument. Epitope combinations which were incorrectly cleaved (i.e. were not cleaved between the two epitopes) were resynthesised with spacer sequences between the CD8+ and CD4+ T-cell epitopes, and their cleavage retested. Appropriate spacer sequences were predicted using the online programme NetChop 3.1 (prediction method: C-term 3.0; threshold: 0.5). Optimal cleavage was identified for the following epitope combinations: C9-H1 (with a spacer of SEQ ID NO: 29); C5-H6 (with a spacer with the sequence A-A-A); C11-H3 (with a spacer with the sequence A-A-A); C4-H4 (with no spacer); and C8-H5 (with no spacer). Following cleavage of these peptides, N-terminal fragments comprising the CTL epitope and C-terminal fragments comprising the Helper epitope were identifiable.

To enhance translocation of the selected CTL epitopes into the endoplasmic reticulum (ER), the algorithm TAPREG (http://imed.med.ucm.es/Tools/tapreg; Diez-Rivero et al. (2010), Proteins 78: 63-72) was used to identify a TTES (Tap Translocation Enhancing Sequence). Based on the TAPREG analysis, the amino acid sequence ARWW (SEQ ID NO: 12) was selected. The SLPs developed as described above were synthesised with the identified TTES at the N-terminus and incubated in vitro and tested by a TAP translocation assay. The TAP translocation assay was performed as described in Neefjes et al., Science 261: 769-771 (1993). The general structure of the designed SLPs is shown in FIG. 6.

### Example 5—Proof of Concept Studies with Specific Conjugates of Invention

Methods

Blood Loop Assay

Blood from donors was taken in an open system and immediately mixed with the anti-coagulant heparin (Leo Pharma AB, Sweden) to a final concentration of 1 IU/ml. All materials in direct contact with the blood were surface-heparinised using the heparin coating kit from Corline (Sweden). Blood and conjugates were applied to heparinised PVC tubings from Corline, which were then sealed using specialised metal connectors, forming loops. The blood loops were rotated on a wheel within a 37° C. incubator. At the end-point sampled blood was mixed with EDTA to a final concentration of 10 mM immediately to stop any ongoing reaction and to prevent clotting of blood. The platelets were counted at 0 and at the end time-point using either a Coulter® Ac-T Diff™ Analyser (Beckman Coulter, Miami, Fla.) or XP-300 (Sysmex, Japan) to ensure that coagulation had not occurred during the experimental procedure and as a response to the reagents added. Plasma was collected and stored at −80° C.

Intracellular Staining & Flow Cytometry Analysis

The intracellular staining of IFNγ and TNFα was performed by adding brefeldin A (Sigma-Aldrich) after 2 hours of circulation of conjugates in the blood loop system. The experiment was terminated after another 4 hours as described for the blood loop system above.

Antibodies for flow cytometry analysis were purchased from Biolegend: anti-CD3 (Clone UCHT1), anti-CD4 (Clone OKT-4), anti-CD8 (clone SK1), anti-CD45RO (Clone UCHL1), anti-IFNγ (Clone 4S.B3) and anti-TNFα (Clone MAb11). Whole blood was stained with cell surface-specific antibodies before red blood cell lysis using FACS lysing solution (BD Biosciences) according to the manufacturer's instructions. The remaining cells were washed and fixed with BD Cytofix/Cytoperm buffer at 4° C. in the dark for 20 minutes. To permeabilize the cells they were first washed and then incubated with Perm/Wash Buffer (BD Biosciences) at RT for 10 minutes. The cells were stained for IFNγ and TNFα for 30 minutes at 4° C. in the dark and subsequently washed in PBS with 1% BSA and 3 mM EDTA (Sigma-Aldrich).

Following staining, the cells were analysed using a Canto II flow cytometer (BD Biosciences) or Cytoflex (Beckman coulter). The cell populations were gated and analysed using FlowJo (Tree Star).

Ethical Considerations

Blood sampling and DTP vaccination of healthy volunteers were approved by the local ethical committee. In short, an 18G gauge needle attached to heparinized tubing was used to draw blood. The blood was collected in a 50 ml surface-heparinized tube and subsequently transferred to the loop tubing and then set to rotate as described above. The DTP vaccination was performed by routine personnel at the hospital using a standard vaccine cocktail.

Results

Blood was taken from donors (prostate cancer patients and healthy volunteers) and the loop assay was performed.

The blood was set to rotate in plastic tubings. Three blood samples from each donor were used: to one of these LUR1-6 conjugates were administered, to another the corresponding naked T-cell epitope-containing antigens (SLP1-6) and to the third saline solution. The LUR1-6 conjugates correspond to Conjugates I-VI as described herein. They were synthesised as described in Example 2; they comprise B-cell epitope-containing peptides of SEQ ID NO: 100 and T-cell epitope containing antigens of SEQ ID NOs: 13-17 and 20, respectively. SLP1-6 correspond to peptides of SEQ ID NOs: 13-17 and 20, respectively.

2 hours after administration of LUR1-6 or SLP1-6, brefeldin was added and after yet another 4 hours the blood was sampled and intracellular staining was performed to analyse cytokine production. Low levels of cytokine production by memory CD8+ T-cells were observed. The donors were then given a booster vaccine comprising TTd (a DTP vaccine) to boost their levels of anti-TTx antibodies. Within approximately 1-2 weeks from this booster, the loop experiment and cytokine production analysis were repeated. Cytokine production in the memory CD8+ T cells was now found in the LUR1-6-treated blood of the two individuals with the highest anti-MTTE-IgG1 levels pre-vaccination (one patient and one healthy volunteer), as shown in FIG. 2A, C. Other cell populations including CD4+CD45RO+ memory T cells (FIG. 2A, C), CD8CD45RO− and CD4CD45RO− (not shown) were unresponsive to treatment with LUR1-6.

These results show that Conjugates I-VI can induce an immune response. The healthy individual in whose blood cytokine production was found was also a male, as such the cytokine production in his blood may be due to previous or ongoing prostatitis that has triggered activation and expansion of auto-reactive T cells.

Blood from a non-DTP-vaccinated patient (donor PMO30) was treated with mouse anti-MTTE IgG2a together with the LUR1-6 mixture, inducing TNFα release by CD8+CD45RO+T memory cells (FIG. 2B, D).

### Example 6—DTP Booster Increases Anti-TTx Antibody Titre in Cancer Patients

The results of Example 5 suggested that administration of a DTP booster vaccine to cancer patients causes an increase in the titres of anti-TTx antibodies, including antibodies which recognise the MTTE of SEQ ID NO: 1 (as is the case in healthy volunteers, Fletcher et al., Journal of Immunology 201(1): 87-97 2018). This was tested experimentally.

Methods

Plasma was obtained from patients as described above in Example 5. Plasma was taken both before a patient received a DTP vaccination and 7-10 days afterwards.

Anti-MTTE antibody titres in plasma from patients (pre- and post-DTP vaccination) were determined using an in-house ELISA. Streptavidin plates (Thermo Scientific) were coated with the peptide of SEQ ID NO: 104, biotinylated at its C-terminus and a scrambled peptide (ETTM) of SEQ ID NO: 103 (also biotinylated at its C-terminus) overnight at 4° C. The plates were washed with PBS (0.05% Tween) and blocked with PBS (10% BSA and 0.05% Tween) for 1 hour at RT. The plasma was serially diluted in PBS (1% BSA and 0.05% Tween), applied to the plates and incubated for 2 hours at RT. MTTE-specific IgM and IgG antibodies were detected with secondary HRP-conjugated antibodies: rabbit anti-human IgG (polyclonal antibody from Dako; diluted 1:4000), anti-IgG1 (Clone HP6070 from Thermo Fisher; diluted 1:500), anti-IgG4 (Clone HP6023 from Thermo Fisher; diluted 1:500) and anti-IgM (polyclonal from Dako; diluted 1:1000). The secondary HRP-conjugated antibodies were diluted in PBS (1% BSA) and incubated on the plates for 1 hour at RT. The reaction was developed with the substrate TMB (Dako) and stopped with 1 M H₂SO₄. The absorbance was read at 450-570 nm using an iMark microplate reader (Bio-Rad).

Results

The results of the analysis are shown in FIG. 3. FIG. 3A shows the titres of IgG antibodies obtained from patients' plasma before and after DTP vaccination; FIG. 3B shows the titres of IgG1 antibodies, FIG. 3C the titres of IgG4 antibodies and FIG. 3D the titres of IgM antibodies.

As shown, a statistically significant increase in the titre of IgG1 antibodies which recognise the MTTE of SEQ ID NO: 1 was seen following administration of a DTP booster to the patients, relative to beforehand. No increase in the titres of IgG4 or IgM antibodies was seen post DTP boost. The data was analysed using paired t-test.

### Example 7—In Vitro Antigen Presentation

Methods

Cells

D1 cells are growth factor-dependent immature dendritic cells (DCs) initially derived from a C57BL/B6 mice. Immature D1 cells were cultured with GM-CSF (20 ng/ml). B3Z is a murine T-cell hybridoma specific for the OVA-derived CD8+ epitope SIINFEKL (SEQ ID NO: 89) in the context of the murine Class I MHC H-2Kb, and which expresses β-galactosidase under the control of the IL-2 promoter (Karttunen et al., PNAS 89(13): 6020-6024, 1992). B3Z cells were cultured in Iscove's Modified Dulbecco's Medium (IMDM) with 10% heat-inactivated FBS, 1% penicillin/streptavidin, 50 μM β-mercaptoethanol and supplemented with Hygromycin B (Invitrogen, Life technologies, Rockville, USA). The generation and culturing of hybridoma cell lines producing mouse anti-MTTE IgG1 and IgG2a antibodies (i.e. antibodies which recognise SEQ ID NO: 1) was performed as described in Fletcher et al. (supra).

In Vitro Antigen Presentation Assay

The antigen presentation assay was performed as previously described (Mangsbo et al, Molecular Immunology 93: 115-124 (2018)). Briefly, immune complexes were pre-formed by incubating the conjugates synthesised in Examples 2 and 3 (which contain the SIINFEKL T-cell epitope recognised by B3Z cells) with an antigen-specific antibody (anti-MTTE IgG1 or IgG2a) at 37° C. for 30 minutes. The immune complexes were incubated with D1 cells (2.5×10⁴/well) for 24 hours, supernatant was removed and subsequently B3Z cells were added and incubated for another 24 hours (5×10⁴/well) at a DC:T-cell ratio of 1:2. The immune complexes were pre-formed at concentrations 3-fold higher than their working concentrations. Addition of the complexes to the D1 cells resulted in their dilution to their working concentrations. The cells were then lysed with a lysing solution (100 mM β-mercaptoethanol, 0.125% IGEPAL CA-630, 9 mM MgCl₂) containing the β-galactosidase substrate chlorophenol red-β D-galactopyranoside (CPRG; 1.8 μg/ml) at 37° C. for 6 hours before the absorbance was measure at 595 nm using an iMark microplate reader (Bio-Rad).

Binding of GMP LUG1-6 Constructs to Human Monoclonal Anti-MTTE IgG1 Antibody

An in-house ELISA was used to confirm binding of GMP-produced LUG1-6 constructs to a recombinant human monoclonal anti-MTTE IgG1 antibody. ELISA plates were coated with 100 μl/well conjugates diluted in Milli-Q water at a range of concentrations (0.000457-1 nmol/ml, a single conjugate per well). The plates were covered and incubated at 4° C. overnight. The plates were subsequently washed four times and blocked with 200 μl/well PBS containing 10% BSA and 0.05% Tween20 and incubated at room temperature (RT) for 1 hour. After washing, the human chimeric anti-MTTE IgG1 antibody (custom made by Evitria AG, Switzerland, >99% monomeric content and <0.1 EU/mg endotoxin), at 0.1 μg/ml in PBS supplemented with 1% BSA and 0.05% Tween20 was added. The plates were washed four times with 250 μl/well PBS containing 0.05% Tween20 and the secondary antibody diluted 1:8000 in PBS supplemented with 1% BSA (anti-human kappa light chain secondary antibody, Thermo Fisher Scientific #A18853) was added to all wells (100 μl/well). After incubation for 1 hour at RT in the dark the plates were washed and 100 μl TMB was added to the wells. The reaction was stopped with 100 μl/well 1 M H₂SO₄ and the absorbance was measured at 450-570 nm wavelength.

Binding of GMP LUG1-6 Constructs to Human Polyclonal Anti-MTTE Antibody

The same in-house ELISA as above was used to confirm binding of GMP-produced LUG1-6 constructs to human polyclonal anti-MTTE antibody from plasma from a human donor previously confirmed to have anti-MTTE antibodies.

ELISA plates were coated with 100 μl/well conjugate diluted in Milli-Q water at a range of concentrations (0.004, 0.03, 0.4 and 1 nmol/ml, a single conjugate per well). The plates were covered and incubated at RT for 2 hours. The plates were then washed 4 times with 250 μl/well PBS containing 0.05% Tween20. The plates were then blocked 3 times with 200 μl/well Superblock T20 (Thermo Scientific) for 5 mins at RT. Plates were washed 4 times with 250 μl/well PBS containing 0.05% Tween20. Donor human plasma was diluted 1:200 in PBS supplemented with 1% BSA and 0.05% Tween20, and 100 μl/well applied to the plates, which were then incubated for 2 hours at RT. Plates were again washed 4 times with 250 μl/well PBS containing 0.05% Tween20, and the secondary antibody diluted 1:8000 in PBS supplemented with 1% BSA (anti-human kappa light chain secondary antibody, Thermo Fisher Scientific #A18853) was added to all wells (100 μl/well). After incubation for 1 hour at RT in the dark the plates were washed and 100 μl TMB was added to the wells. The reaction was stopped with 100 μl/well 1 M H₂SO₄ and the absorbance was measured at 450-570 nm wavelength.

Results

DC1 dendritic cells were incubated with immune complexes formed from conjugates synthesised according to Examples 2 and 3. These conjugates are essentially identical, except that the conjugates synthesised according to Example 2 contain intact succinimide rings, whereas the rings of the conjugates synthesised according to Example 3 are opened. The results of these experiments are shown in FIG. 4, in which a higher absorbance at 595 nm indicates a higher level of T-cell activation.

The results obtained with presentation of antigen from conjugates with intact rings are shown in FIG. 4A; the results obtained with presentation of antigen from conjugates with opened rings are shown in FIG. 4B. As can be seen, both conjugates were able to activate B3Z T-cells in the antigen presentation assay, though the conjugate with opened succinimide rings drove a greater level of T-cell activation. Conjugates with open rings were also confirmed to bind anti-MTTE antibodies as analysed by ELISA (both recombinant monoclonal human IgG1 antibody, FIG. 7A; and polyclonal human donor antibody, FIG. 7B).

### Example 8—HLA Profile of Responders and Memory CD8 T-Cell Responses to an Individual Construct in One Patient and One Healthy Individual

The two individuals whose blood showed an increased response to the mix of LUR1-6 conjugates following DTP booster vaccination in Example 5 were analysed to determine their HLA profiles, and thus which conjugate(s) they may have been responding to. In addition one patient that did not receive a DTP booster but that displayed a response when rabbit anti-MTTE antibodies were given in conjunction with the constructs was assessed. However this donor that was also HLA profiled, displayed blood clotting during sampling and as such the experimental plan was not fully executed and all loops were not run. Thus this patient was removed from the data analysis and is not displayed below:

Based on the peptide's CD8 epitope and donor's HLA-type class I the analysed patient can respond to LUG2, 3 and 6 and the healthy individual can respond to LUG2, 3, 5 and 6.

LUG1=SEQ ID NO: 13; LUG2=SEQ ID NO: 14; LUG3=SEQ ID NO: 15; LUG4=SEQ ID NO: 16; LUG5=SEQ ID NO: 17; LUG6=SEQ ID NO: 20.

| | HLA-TYPE | HLA-TYPE |
|---|---|---|
| DONOR | CLASS I | CLASS II |
| PATIENT | HLA*A2:01 | HLA-DRB1*04 |
| | | HLA-DRB1*13 |
| HEALTHY | HLA*A2:01 | HLA-DRB1*15 |
| INDIVIDUAL | HLA*A03:01:01 | |

Methods

The loop assay was performed as in Example 5 but with the following loops assessed per individual:
1. Vehicle (NaCl 0.9%)
2. anti-MTTE IgG2a (40 μg/ml)
3. LUG1-6 (6×125 nM)
4. LUG1-6 (6×125 nM)+anti-MTTE IgG2a (40 μg/ml)
5. LUG1 (125 nM)
6. LUG1 (125 nM)+anti-MTTE IgG2a (40 μg/ml)
7. LUG2 (125 nM)
8. LUG2 (125 nM)+anti-MTTE IgG2a (40 μg/ml)
9. LUG3 (125 nM)
10. LUG3 (125 nM)+anti-MTTE IgG2a (40 μg/ml)
11. LUG4 (125 nM)
12. LUG4 (125 nM)+anti-MTTE IgG2a (40 μg/ml)
13. LUG5 (125 nM)
14. LUG5 (125 nM)+anti-MTTE IgG2a (40 μg/ml)
15. LUG6 (125 nM)
16. LUG6 (125 nM)+anti-MTTE IgG2a (40 μg/ml)

An average value was calculated from each duplicate loop, regardless of whether the loop was spiked with rabbit anti-MTTE antibodies or not. Both were used in the analysis as values with or without anti-MTTE spiked loops did not differ and as endogenous antibodies are present to the MTTE in these individuals from the previous DTP vaccination. The mean value of loop 1 and 2 was used as the background vehicle value. The fold change was calculated by the mean value of the compound exposed loop divided by the mean value of the background vehicle sample.

The results of the fold increase in recall response of IFNγ-producing CD8+ memory (CD45RO+) T-cells is shown in FIG. 5. CD4 responses were not detectable at the tested time point (not shown).

Results

The results show that the patient responded with IFNγ production to both the mix of the conjugates and to individual conjugates and that the response to the individual conjugates matches the expected response based on the HLA type. The healthy individual did not display a response above background during this analysis, possibly reflecting that any inflammatory cause that led to a spike in auto-immune T-cells in circulation at the first analysis (FIG. 2) was not present and as such those auto-reactive T-cells had vanished.

The patient also displayed a response to LUG5 which cannot be predicted based on the HLA profile of the selected CD8 epitope. However it cannot be excluded that the CD4 epitope harbours an HLA class I epitope that the patient responds to. Via the IEBD analysis resource Consensus tool (Kim et al., Protein Sci 12: 1007-1017 (2003)) the sequence YTLRVDCTPL (SEQ ID NO: 97) in the CD4 epitope in LUG5 was predicted to bind to HLA-A*02:01 with a low percentile rank.

LUG5 also displayed elevated IFNγ responses in the CD4+CD45RO negative population (not shown).

Whenever the abbreviation LUG is used, it means that a construct is of GMP quality, and whenever the abbreviation LUR is used, it means that a construct is made for research purposes. Structurally each construct LUG1, LUG2, LUG3, LUG4, LUG5 and LUG6 corresponds to the construct LUR 1, LUR2, LUR3, LUR4 LUR5 5 and LUR6.

### Example 9—Testing of Conjugates in Mice

Methods

Evaluation of Epitope-Specific T Cell Responses in Humanised HLA-DR4 Mice

Female HLA-DR4 transgenic mice on a C57/B16 background (12 weeks old at the start of the study) were acquired from Taconic (Germantown, Md., USA). HLA-DR4 animals were administered a LUG2 construct (20 μg or 5 μg) subcutaneously at the tail base followed by a boost two weeks later. A week later the mice were sacrificed, and the spleens were collected for generation of single cell suspensions for analysis by ELISPOT as described below. Heart bleed was performed to analyze anti-MTTE titers after LUG2 exposure. Tail vein-sampled HLA-DR4 animals that had not been exposed to LUG2 were used as controls for baseline titre assessment (unexposed animals).

Evaluation of Immune Responses

Antibody titres against the MTTE were determined using an in-house ELISA. Streptavidin plates (Thermo Scientific) were coated with the peptide of SEQ ID NO: 104, biotinylated at its C-terminus, overnight at 4° C. The plates were washed with PBS (0.05% Tween) and blocked with PBS (10% BSA and 0.05% Tween) for 1 hour at RT. The mouse serum was serially diluted in PBS (1% BSA and 0.05% Tween), applied to the plates and incubated for 2 hours at RT. Mouse MTTE-specific IgG antibodies were detected with secondary HRP-conjugated antibody: goat anti-mouse IgG (polyclonal antibody from Dako; diluted 1:4000). The secondary HRP-conjugated antibody was diluted in PBS (1% BSA) and incubated on the plates for 1 hour at RT. The reaction was developed with the substrate TMB (Dako) and stopped with 1 M H₂SO₄. The absorbance was read at 450-570 nm using an iMark microplate reader (Bio-Rad).

The immunogenicity of the HLA-DR4 epitope was assessed by stimulating splenocytes with SLPs containing the embedded HLA-DR4 sequence. This was performed using an ex vivo IFNγ ELISpot assay (ELISpot kit for mouse IFNγ/3321-2A, Mabtech, Stockholm, Sweden). The LUG2 SLP with the TAP sequence has the amino acid sequence set forth in SEQ ID NO: 14, and the LUG2 SLP without the TAP sequence is set forth in SEQ ID NO: 107; both contain the embedded HLA-DR4 sequence. One day before spleens were harvested, 96-well ELISpot plates (Millipore) for the IFN-γ ELISpot assay were pre-coated with capture antibody according to the manufacturer's protocol. After 5 washes with PBS/Tween and blocking for a minimum of 30 min with T cell medium including RPMI 1640 (Life Technologies/Thermo Fisher Scientific), containing 1% w/v L-Glutamine (SLS/Lonza), 10% v/v FBS (Fisher/GE Healthcare), 2% HEPES (SLS/Lonza), 0.1% v/v Fungizone (Promega), 0.5×10⁶/well freshly isolated splenocytes were seeded in triplicate into the plate along with 100 μl of the respective SLPs at a final concentration of 10 μg/ml. The cells were then incubated at 37° C. in a 5% CO₂ incubator for 48 hours, and the plates then washed 5 times with DPBST. 50 μl/well biotinylated detection antibody (1/1000 dilution) against mouse IFNγ was then added, and the plates incubated for 2 hours at room temperature. Plates were then washed 5 times with DPBST, followed by the addition of 50 μl/well streptavidin alkaline phosphatase (1/1000 dilution). Plates were then incubated for 1 h 30 min at room temperature. After incubation, plates were washed again 6 times with DPBST and then 50 μl/well development solution (BCIP/NBT, BioRad) was added. The plates were left in the dark at room temperature until spots could be seen. Once spots developed, the reaction was stopped by rinsing the plates with tap water. Plates were then left to dry and the spots were quantified using an ELISpot plate reader (Cellular Technology Limited, Shaker Heights, Ohio, USA). SEB, the staphylococcal enterotoxin-B (at 2.5 μg/ml) was used as positive control, and unstimulated splenocytes (cells alone) were used as a negative control for every ELISpot assay. All experiments were performed in triplicate. Animals were scored as having a positive reaction when the number of spots in the cells-alone wells did not reach more than 20 and when the response in the peptide-containing wells was at least twice that of the standard deviation of the mean of the control wells.

Results

Evaluation of cytokine-expressing T cells in the human whole blood loop assay identified a small fraction of both healthy individuals and prostate cancer patients that responded with IFNγ and/or TNFα expression upon formation of ICs with the LUR/LUG 1-6 constructs. However, this assay was limited by the low frequency of epitope-specific T cells in the human blood and the lack of tetramers/multimers that could increase the sensitivity of the method. Therefore, to address in vivo priming and expansion of epitope-specific T cells commercial HLA-DR4 mice were used. As LUG2 includes an HLA-DR4 restricted PSMA epitope it was possible to expose animals to LUG2 conjugates and evaluate CD4+ T cell priming. HLA-DR4 mice received a prime/boost vaccination schedule with the LUG2 constructs. From serum collected from the LUG2 exposed animal and un-exposed animals as controls we identified that mice exposed to LUG2 increased their anti-MTTE antibody titers. Upon treatment of splenocytes from the LUG2 vaccinated animals with the SLP contained in the LUG2 construct (UV02, SEQ ID NO: 14) or the SLP without the TAP ARWW sequence (UV08, SEQ ID NO: 107), an increased number of IFN-γ producing T cells was noted (FIG. 8 shows the results obtained from mice vaccinated with 20 μg LUG2; a similar pattern of results was obtained from mice vaccinated with 5 μg LUG2—data not shown).

### Example 10—Conjugate Safety

Methods

Cytokine and Complement Analysis in Plasma from Boostrix-Vaccinated Patients

Approximately two weeks after vaccination with the TDP vaccine Boostrix (GSK, Brentford, UK), blood was collected from five healthy individuals and five prostate cancer patients.

For evaluation of infusion reactions with regards to cytokine release and complement activation, the blood was treated with 3 different concentrations of the TENDU vaccine mixed constructs LUG1-6 using 0.05 μg/ml, 0.5 μg/ml and 2.5 μg/ml of each individual construct. Plasma harvested after 0 and 4 hours in the blood loop assay was used for concentration determination of IFN-γ, IL-1β, IL-2, IL-6, IL-8, IL-10 and TNF-α using Mesoscale V-plex kit (MSD Discovery®, Kenilworth, N.J., USA) according to the manufacturer's instructions. Lower limit of detection (LLOD) was calculated using MSD software and defined as 2.5×SD above the zero calibrator. Upper limit of detection (ULOD) was calculated using MSD software from the signal value of the Standard-1. Lower and upper limit of quantifications (LLOQ and ULOQ) are verified using MSD and calculated from the standard curve and percentage recovery of diluent standards with precision of 20% and accuracy 80-120%.

Plasma harvested after 0 and 15 minutes in the blood loop assay was analysed for complement activation (C3a and C5a) with ELISA kits from Hycult Biotech (Uden, Netherlands) according to the manufacturer's instructions.

Rabbit Toxicity

The TENDU vaccine was tested for toxicity in male rabbits by Meditox (Konerovice, Czech Republic). The rabbits were subcutaneously vaccinated four times (with two-week intervals) with tetanus toxoid vaccine (Equip® T vet.≥30 IU/ml, Orion Pharma Animal Health, Danderyd, Sweden) to generate TTd seropositive animals. After another two weeks the rabbits were subcutaneously vaccinated four times (with two-week intervals) with TENDU at low (10 μg/construct, n=5), intermediate (100 μg/construct, n=5) or high (240 μg/construct, n=8) dose. The two control groups were rabbits only receiving the tetanus vaccination (n=5) and rabbits that only received the high dose of TENDU (n=5). Clinical observations were made such as body weight, body temperature, food consumption, ophthalmoscopy, blood analysis, serum chemistry, urine analysis and a pathological examination.

Blood samples were collected in K3 EDTA tubes on week 15 before TENDU administration and post-TENDU administration at 4 h and 24 h. Blood samples were centrifuged (3500 rpm for 10 min, at 4° C.). The plasma was collected and stored at −20° C. until analysis with ELISA.

ELISA for Cytokine Detection in Rabbit Plasma

The following ELISA kits were used for analysis of cytokines in rabbit plasma: RayBio Rabbit IL-8 (cat. no ELL-IL-8-1), RayBio IL-1β (cat. no ELL-IL1b-1) and RayBio Rabbit IFNγ (cat. no ELL-IFNg-1) (Norcross, Ga., USA). Cytokine analysis was performed according to manufacturer's instructions.

Results

The safety of the vaccine constructs was evaluated using a blood loop system with blood samples from both healthy individuals and prostate cancer patients vaccinated with Boostrix. We assessed cytokine release and complement activation at three doses. The highest dose of each conjugate administrated was 240 μg. In humans this dose would lead to a Cmax of approximately 0.024 μg/ml per conjugate with an estimation of that a body contains 10 L blood/extracellular liquid. Complement activation in response to immune complex formation can lead to release of C3a and C5a components which act as anaphylatoxins and increase inflammatory response. We analysed the concentration and production of the cleaved complement components C5a and C3a (FIG. 9A-B). C3a concentration increased slightly in response to both 0.5 μg/ml and 2.5 μg/ml LUG1-6 constructs in healthy individuals and prostate cancer patients, concentrations that are above any expected Cmax concentration from subcutaneous administration of the conjugates. C5a concentration was similarly increased only upon treatment with 2.5 μg/ml of each LUG1-6 construct. For the lowest dose, in the range of any expected systemic exposure range, no complement activation was detected. As expected alemtuzumab, an antibody that is known to lead to complement fixation, led to increased concentrations of both C3a and C5a in both prostate cancer patients and healthy individuals. Infusion of biotherapeutics in blood can through different mechanisms induce cytokine release, and therefore we analysed the production of a set of cytokines after stimulation with the LUG1-6 constructs. Alemtuzumab, an antibody known to induce cytokine release, led to a significant increase in production of cytokines IL-8 (FIG. 9C), IFNγ, IL-6 IL-1bTNFα (FIG. 9F-I) and IL-10 (data not shown). LUG1-6 constructs only led to a notable elevation of IL-8 at the highest concentration of 2.5 μg/ml of each vaccine construct, while the remaining cytokines IFNγ, IL-6 and TNFα were not affected by treatment with the TENDU constructs at any of the concentrations analysed. Additionally, IL-2, IL-10 and IL-1β production was not affected by any of the TENDU concentrations used (data not shown).

Safety of TENDU was also assessed in vivo, in either tetanus toxoid seronegative or seropositive male rabbits. Rabbits were vaccinated four times with low, intermediate or high dose of TENDU and no clinical signs of toxicity were observed in any of the groups. The subcutaneous injections did not induce any local adverse reactions and there was no effect on body weight, food consumption, or body temperature of the rabbits in the study. To evaluate possible risks due to cytokine release after subcutaneous administration of TENDU and since IL-8 was released upon direct exposure of blood to TENDU at 2.5 μg/ml of each LUG construct, plasma collected from rabbits was analysed for IFN-γ, IL-8 and IL-1b. IFN-γ was undetectable in the majority of the samples without any increase in the concentration noticed after TENDU administration (FIG. 9E) however IL-8 concentrations in plasma from DTP-vaccinated rabbits were slightly increased over time (up to 24 h) in some of the animals in the different dose groups without a clear association with administration of high TENDU dose (FIG. 9D). IL-1β was undetectable at all time points and TENDU doses analysed (data not shown).

1. A conjugate comprising at least one B-cell epitope-containing peptide conjugated to a T-cell epitope-containing antigen, wherein:
(i) said at least one B-cell epitope-containing peptide comprises a minimal tetanus toxoid epitope (MTTE), said MTTE comprising:
(a) an amino acid sequence of at least 10 amino acids which are contiguous in SEQ ID NO: 22 and comprise the amino acid sequence GITELKKL set forth in SEQ ID NO: 23; or
(b) an amino acid sequence with at least 70% sequence identity to an amino acid sequence of (a);
wherein said B-cell epitope-containing peptide is not the complete tetanus toxin beta chain;
(ii) said T-cell epitope-containing antigen is a polypeptide comprising from N-terminus to C-terminus:
(a) a translocation peptide;
(b) a CD8+ T-cell cancer epitope; and
(c) a CD4+ T-cell cancer epitope;
(iii) the N-terminus of said T-cell epitope-containing antigen is conjugated to said B-cell epitope-containing peptide; and wherein
(iv) the conjugation of the at least one B-cell epitope-containing peptide and the T-cell epitope-containing antigen may be direct or indirect.

2. The conjugate of claim 1, further comprising a proteasome cleavage site positioned between the CD8+ T-cell epitope and the CD4+ T-cell epitope.

3. The conjugate of claim 2, wherein the proteasome cleavage site is provided by a spacer.

4. The conjugate of any one of claims 1 to 3, comprising a spacer sequence between the B-cell epitope-containing peptide and the T-cell epitope-containing antigen, said spacer being C-terminal to the MTTE.

5. The conjugate of any one of claims 1 to 4, wherein the translocation peptide comprises the amino acid sequence set forth in SEQ ID NO: 12, or an amino acid sequence with at least 75% sequence identity thereto.

6. The conjugate of any one of claims 1 to 5, wherein the CD8+ T-cell cancer epitope is a prostate cancer epitope.

7. The conjugate of any one of claims 1 to 6, wherein the CD4+ T-cell cancer epitope is a prostate cancer epitope.

8. The conjugate of any one of claims 1 to 7, wherein the CD8+ T-cell cancer epitope comprises an 8-15 amino acid fragment of SEQ ID NO: 24 or of SEQ ID NO: 25, or an amino acid sequence with at least 65% sequence identity to any such fragment.

9. The conjugate of claim 8, wherein the CD8+ T-cell cancer epitope comprises an 8-9 amino acid fragment of SEQ ID NO: 24 or of SEQ ID NO: 25, or an amino acid sequence with at least 65% sequence identity to any such fragment.

10. The conjugate of claim 8 or 9, wherein the CD8+ T-cell cancer epitope comprises an amino acid sequence selected from any one of SEQ ID NOs: 2, 3, 4, 5 and 6, or an amino acid sequence with at least 65% sequence identity thereto.

11. The conjugate of any one of claims 1 to 10, wherein the CD4+ T-cell cancer epitope comprises an 11-30 amino acid fragment of SEQ ID NO: 24 or of SEQ ID NO: 25, or an amino acid sequence with at least 75% sequence identity to any such fragment.

12. The conjugate of claim 11, wherein said CD4+ T-cell cancer epitope comprises a 12-18 amino acid fragment of SEQ ID NO: 24 or of SEQ ID NO: 25, or an amino acid sequence with at least 75% sequence identity to any such fragment.

13. The conjugate of claim 11 or 12, wherein the CD4+ T-cell cancer epitope comprises an amino acid sequence selected from any one of SEQ ID NOs: 7, 8, 9, 10 and 11, or an amino acid sequence with at least 75% sequence identity thereto.

14. The conjugate of claim 10 or 13, being Conjugate I, wherein the CD8+ T-cell cancer epitope comprises the amino acid sequence set forth in SEQ ID NO: 2 or an amino acid sequence with at least 65% sequence identity thereto, and the CD4+ T-cell cancer epitope comprises the amino acid sequence of SEQ ID NO: 7 or an amino acid sequence with at least 75% sequence identity thereto.

15. The conjugate of claim 14, wherein the T-cell epitope-containing antigen comprises the amino acid sequence set forth in SEQ ID NO: 13 or an amino acid sequence with at least 70% sequence identity thereto.

16. The conjugate of claim 10 or 13, being Conjugate II, wherein the CD8+ T-cell cancer epitope comprises the amino acid sequence set forth in SEQ ID NO: 3 or an amino acid sequence with at least 65% sequence identity thereto, and the CD4+ T-cell cancer epitope comprises the amino acid sequence of SEQ ID NO: 8 or an amino acid sequence with at least 75% sequence identity thereto.

17. The conjugate of claim 16, wherein the T-cell epitope-containing antigen comprises the amino acid sequence set forth in SEQ ID NO: 14 or an amino acid sequence with at least 70% sequence identity thereto.

18. The conjugate of claim 10 or 13, being Conjugate III, wherein the CD8+ T-cell cancer epitope comprises the amino acid sequence set forth in SEQ ID NO: 4 or an amino acid sequence with at least 65% sequence identity thereto, and the CD4+ T-cell cancer epitope comprises the amino acid sequence of SEQ ID NO: 9 or an amino acid sequence with at least 75% sequence identity thereto.

19. The conjugate of claim 18, wherein the T-cell epitope-containing antigen comprises the amino acid sequence set forth in SEQ ID NO: 15 or an amino acid sequence with at least 70% sequence identity thereto.

20. The conjugate of claim 10 or 13, being Conjugate IV, wherein the CD8+ T-cell cancer epitope comprises the amino acid sequence set forth in SEQ ID NO: 5 or an amino acid sequence with at least 65% sequence identity thereto, and the CD4+ T-cell cancer epitope comprises the amino acid sequence of SEQ ID NO: 10 or an amino acid sequence with at least 75% sequence identity thereto.

21. The conjugate of claim 20, wherein the T-cell epitope-containing antigen comprises the amino acid sequence set forth in SEQ ID NO: 16 or an amino acid sequence with at least 70% sequence identity thereto.

22. The conjugate of claim 10 or 13, being Conjugate V, wherein the CD8+ T-cell cancer epitope comprises the amino acid sequence set forth in SEQ ID NO: 6 or an amino acid sequence with at least 65% sequence identity thereto, and the CD4+ T-cell cancer epitope comprises the amino acid sequence of SEQ ID NO: 11 or an amino acid sequence with at least 75% sequence identity thereto.

23. The conjugate of claim 22, wherein the T-cell epitope-containing antigen comprises the amino acid sequence set forth in SEQ ID NO: 17 or an amino acid sequence with at least 70% sequence identity thereto.

24. The conjugate of any one of claims 1 to 23, wherein the MTTE comprises the amino acid sequence set forth in SEQ ID NO: 1 or an amino acid sequence with at least 70% sequence identity thereto.

25. The conjugate of any one of claims 1 to 23, wherein the MTTE comprises the amino acid sequence set forth in any one of SEQ ID NOs: 30-86 or an amino acid sequence with at least 70% sequence identity thereto.

26. The conjugate of any one of claims 1 to 25, wherein the N-terminus of the T-cell epitope-containing antigen is conjugated to the C-terminal amino acid of said at least one B-cell epitope-containing peptide.

27. The conjugate of any one of claims 1 to 26, wherein the B-cell epitope-containing peptide further comprises a cysteine residue.

28. The conjugate of any one of claim 21 or 23-25, wherein the at least one B-cell epitope-containing peptide comprises the amino acid sequence set forth in SEQ ID NO: 21 or SEQ ID NO: 100, or an amino acid sequence with at least 70% sequence identity to SEQ ID NO: 21 or SEQ ID NO: 100.

29. The conjugate of any one of claims 1 to 28, wherein said conjugate comprises at least three B-cell epitope-containing peptides.

30. The conjugate of any one of claims 27-29, wherein said conjugate has a chemical structure selected from: 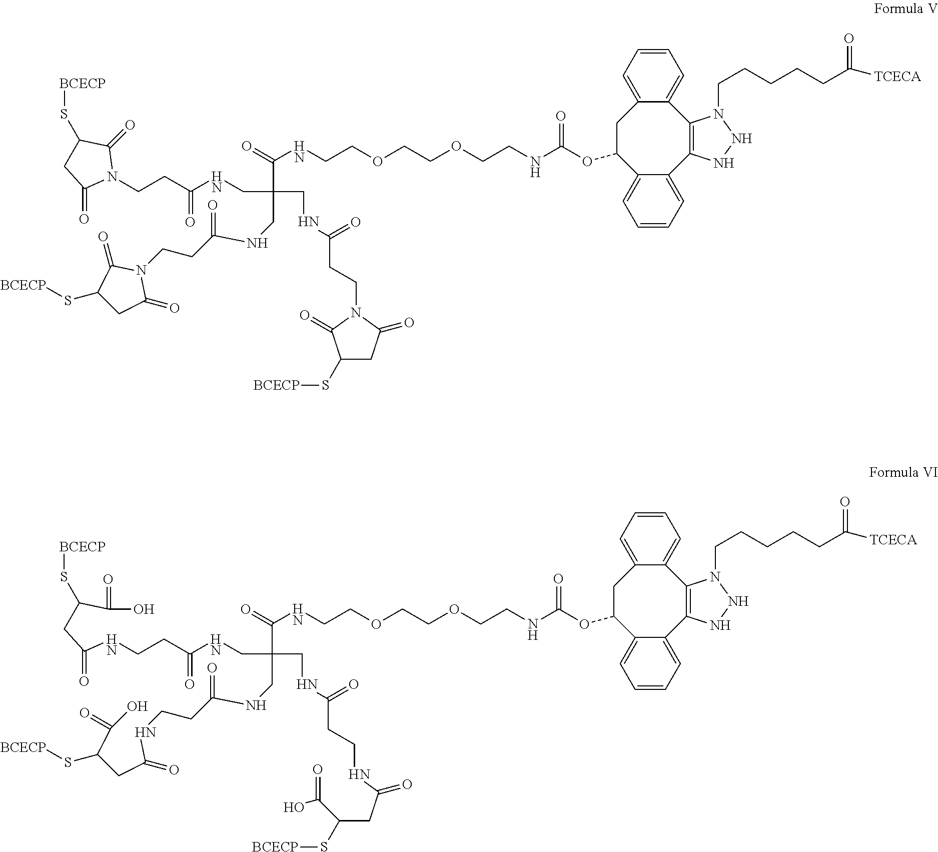
or an isomer or enantiomer of Formula VI;
wherein BCECP indicates a B-cell epitope-containing peptide and TCECA indicates a T-cell epitope containing antigen, and in each of said structures each sulphur atom linking a B-cell epitope-containing peptide to an open or closed succinimide ring is from the thiol group of a cysteine residue of the attached B-cell epitope-containing peptide, and the linkage of the T-cell epitope-containing antigen to the chemical structure is a peptide bond to the N-terminus of the T-cell epitope-containing antigen.

31. The conjugate of claim 30, wherein the B-cell epitope-containing peptide comprises the amino acid sequence set forth in SEQ ID NO: 21, and the T-cell epitope-containing antigen comprises the amino acid sequence set forth in SEQ ID NO: 13, SEQ ID NO: 14, SEQ ID NO: 15, SEQ ID NO: 16, SEQ ID NO: 17 or SEQ ID NO: 20.

32. A conjugate, being Conjugate VI, comprising at least one B-cell epitope-containing peptide conjugated to a T-cell epitope-containing antigen, wherein: said B-cell epitope-containing peptide is as defined in any one of claims 1, 24-25 or 27-28; said T-cell epitope-containing antigen is a peptide comprising a 20-35 amino acid fragment of SEQ ID NO: 18, or an amino acid sequence with at least 70% sequence identity to such a fragment; and the N-terminus of said antigen is conjugated to said B-cell epitope-containing peptide.

33. The conjugate of claim 32, wherein said T-cell epitope-containing antigen comprises the amino acid sequence set forth in SEQ ID NO: 19 or an amino acid sequence with at least 65% sequence identity thereto.

34. The conjugate of claim 33, wherein said T-cell epitope-containing antigen comprises the amino acid sequence set forth in SEQ ID NO: 20 or an amino acid sequence with at least 70% sequence identity thereto.

35. The conjugate of any one of claims 32 to 34, wherein:
(a) said conjugate comprises at least three B-cell epitope-containing peptides;
(b) said conjugate has a chemical structure as defined in claim 30; and/or
(c) in said conjugate the N-terminus of the T-cell epitope-containing antigen is conjugated to the C-terminus of the B-cell epitope-containing peptide.

36. A vaccine composition comprising at least one conjugate as defined in any one of claims 1 to 35.

37. The vaccine composition of claim 36, said vaccine composition comprising one or more of Conjugate I, II, III, IV or V of claims 13 to 30.

38. The vaccine composition of claim 37, said vaccine composition comprising Conjugates I, II, III, IV and V of claims 13 to 30.

39. The vaccine composition of any one of claims 36 to 38, further comprising Conjugate VI of claims 32 to 35.

40. The vaccine composition of any one of claims 36 to 39, wherein each B-cell epitope-containing peptide of each conjugate in the vaccine composition is identical.

41. The vaccine composition of claim 40, said vaccine composition comprising Conjugate I, Conjugate II, Conjugate IV and Conjugate V of claims 14 to 17 and 20 to 23.

42. The vaccine composition of claim 40, said vaccine composition comprising Conjugate I, Conjugate III and Conjugate V of claims 14 to 15, 18 to 19 and 22 to 23.

43. The vaccine composition of any one of claims 36 to 42, further comprising one or more pharmaceutically-acceptable diluents, carriers or excipients.

44. A conjugate as defined in any one of claims 1 to 35, or a vaccine composition as defined in any one of claims 36 to 43, for use in therapy.

45. A conjugate as defined in any one of claims 1 to 35, or a vaccine composition as defined in any one of claims 36 to 43, for use in prevention or treatment of cancer.

46. The conjugate or vaccine composition for use according to claim 45, wherein said cancer is prostate cancer.

47. A method for the prevention or treatment of cancer in a subject in need of such prevention or treatment, comprising administering to said subject a therapeutically effective amount of a conjugate as defined in any one of claims 1 to 35 or a vaccine composition as defined in any one of claims 36 to 43.

48. Use of a conjugate as defined in any one of claims 1 to 35, or a vaccine composition as defined in any one of claims 36 to 42, in the manufacture of a medicament for use in the prevention or treatment of cancer.

49. A polypeptide comprising or consisting of an amino acid sequence set forth in any one of SEQ ID NOs: 13-17, or an amino acid sequence with at least 70% sequence identity thereto, wherein said polypeptide comprises from N-terminus to C-terminus:
(a) a translocation peptide;
(b) a CD8+ T-cell cancer epitope; and
(c) a CD4+ T-cell cancer epitope;
wherein a proteasome cleavage site is optionally present between said CD8+ T-cell cancer epitope and said CD4+ T-cell cancer epitope, optionally wherein said cleavage site is provided by a spacer;
wherein said translocation peptide is able to mediate TAP-driven transport of said polypeptide or said CD8+ T-cell cancer epitope into the endoplasmic reticulum of a host cell.

50. A nucleic acid molecule comprising or consisting of a nucleotide sequence encoding a polypeptide as defined in claim 49.

51. A construct or vector comprising a nucleic acid molecule as defined in claim 50.

52. A kit comprising a vaccine composition as defined in any one of claims 36 to 43, and a second therapeutically active agent.


SEQUENCE LISTING (110 sequences)

SEQ ID NO: 1 - protein, 18 aa, Clostridium tetani
FIGITELKKLESKINKVF

SEQ ID NO: 2 - protein, 9 aa, Homo sapiens
NYARTEDFF

SEQ ID NO: 3 - protein, 9 aa, Homo sapiens
LLHETDSAV

SEQ ID NO: 4 - protein, 9 aa, Homo sapiens
SLSLGFLFL

SEQ ID NO: 5 - protein, 9 aa, Homo sapiens
GMPEGDLVY

SEQ ID NO: 6 - protein, 9 aa, Homo sapiens
KVFRGNKVK

SEQ ID NO: 7 - protein, 15 aa, Homo sapiens
GQDLFGIWSKVYDPL

SEQ ID NO: 8 - protein, 15 aa, Homo sapiens
RQIYVAAFTVQAAAE

SEQ ID NO: 9 - protein, 15 aa, Homo sapiens
GKVFRGNKVKNAQLA

SEQ ID NO: 10 - protein, 15 aa, Homo sapiens
TGNFSTQKVKMHIHS

SEQ ID NO: 11 - protein, 15 aa, Homo sapiens
NYTLRVDCTPLMYSL

SEQ ID NO: 12 - protein, 4 aa, Homo sapiens
ARWW

SEQ ID NO: 13 - protein, 34 aa, Artificial Sequence
  Conjugate I SLP
ARWWNYARTEDFFQQQPPPGQDLFGIWSKVYDPL

SEQ ID NO: 14 - protein, 31 aa, Artificial Sequence
  Conjugate II SLP
ARWWLLHETDSAVAAARQIYVAAFTVQAAAE

SEQ ID NO: 15 - protein, 31 aa, Artificial Sequence
  Conjugate III SLP
ARWWSLSLGFLFLAAAGKVFRGNKVKNAQLA

SEQ ID NO: 16 - protein, 28 aa, Artificial Sequence
  Conjugate IV SLP
ARWWGMPEGDLVYTGNFSTQKVKMHIHS

SEQ ID NO: 17 - protein, 28 aa, Artificial Sequence
  Conjugate V SLP
ARWWKVFRGNKVKNYTLRVDCTPLMYSL

SEQ ID NO: 18 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 19 - protein, 9 aa, Homo sapiens
LAMPFATPM

SEQ ID NO: 20 - protein, 27 aa, Homo sapiens
GARGPESRLLEFYLAMPFATPMEAELA

SEQ ID NO: 21 - protein, 29 aa, Artificial Sequence
  B Cell Epitope Containing Peptide
FIGITELKKLESKINKVFAAKYARVRAKC

SEQ ID NO: 22 - protein, 858 aa, Clostridium tetani
SLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 23 - protein, 8 aa, Clostridium tetani
GITELKKL

SEQ ID NO: 24 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 25 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 26 - protein, 1314 aa, Clostridium tetani
PITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 27 - protein, 456 aa, Clostridium tetani
PITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTA

SEQ ID NO: 28 - protein, 10 aa, Artificial Sequence
  Spacer Sequence
AAKYARVRAK

SEQ ID NO: 29 - protein, 6 aa, Artificial Sequence
  Conjugate I Spacer Sequence
QQQPPP

SEQ ID NO: 30 - protein, 12 aa, Clostridium tetani
FIGITELKKLES

SEQ ID NO: 31 - protein, 12 aa, Clostridium tetani
GITELKKLESKI

SEQ ID NO: 32 - protein, 14 aa, Clostridium tetani
SKFIGITELKKLES

SEQ ID NO: 33 - protein, 14 aa, Clostridium tetani
FIGITELKKLESKI

SEQ ID NO: 34 - protein, 16 aa, Clostridium tetani
ANSKFIGITELKKLES

SEQ ID NO: 35 - protein, 16 aa, Clostridium tetani
SKFIGITELKKLESKI

SEQ ID NO: 36 - protein, 18 aa, Clostridium tetani
IKANSKFIGITELKKLES

SEQ ID NO: 37 - protein, 18 aa, Clostridium tetani
ANSKFIGITELKKLESKI

SEQ ID NO: 38 - protein, 20 aa, Clostridium tetani
QYIKANSKFIGITELKKLES

SEQ ID NO: 39 - protein, 20 aa, Clostridium tetani
IKANSKFIGITELKKLESKI

SEQ ID NO: 40 - protein, 22 aa, Clostridium tetani
QYIKANSKFIGITELKKLESKI

SEQ ID NO: 41 - protein, 20 aa, Clostridium tetani
FIGITELKKLESKINKVFST

SEQ ID NO: 42 - protein, 19 aa, Clostridium tetani
FIGITELKKLESKINKVFS

SEQ ID NO: 43 - protein, 18 aa, Clostridium tetani
FIGITELKKLESKINKVF

SEQ ID NO: 44 - protein, 17 aa, Clostridium tetani
FIGITELKKLESKINKV

SEQ ID NO: 45 - protein, 16 aa, Clostridium tetani
FIGITELKKLESKINK

SEQ ID NO: 46 - protein, 15 aa, Clostridium tetani
FIGITELKKLESKIN

SEQ ID NO: 47 - protein, 14 aa, Clostridium tetani
IGITELKKLESKIN

SEQ ID NO: 48 - protein, 14 aa, Clostridium tetani
GITELKKLESKINK

SEQ ID NO: 49 - protein, 14 aa, Clostridium tetani
  MOD_RES (8)..(8): FORMYLATION
FIGITELKKLESKI

SEQ ID NO: 50 - protein, 14 aa, Clostridium tetani
  MOD_RES (9)..(9): FORMYLATION
FIGITELKKLESKI

SEQ ID NO: 51 - protein, 14 aa, Clostridium tetani
  MOD_RES (13)..(13): FORMYLATION
FIGITELKKLESKI

SEQ ID NO: 52 - protein, 14 aa, Clostridium tetani
  MOD_RES (8)..(9): FORMYLATION
FIGITELKKLESKI

SEQ ID NO: 53 - protein, 14 aa, Clostridium tetani
  MOD_RES (9)..(9): FORMYLATION
  MOD_RES (13)..(13): FORMYLATION
FIGITELKKLESKI

SEQ ID NO: 54 - protein, 18 aa, Artificial Sequence
  Modified MTTE
AIGITELKKLESKINKVF

SEQ ID NO: 55 - protein, 18 aa, Artificial Sequence
  Modified MTTE
FAGITELKKLESKINKVF

SEQ ID NO: 56 - protein, 18 aa, Artificial Sequence
  Modified MTTE
FIAITELKKLESKINKVF

SEQ ID NO: 57 - protein, 18 aa, Artificial Sequence
  Modified MTTE
FIGITALKKLESKINKVF

SEQ ID NO: 58 - protein, 18 aa, Artificial Sequence
  Modified MTTE
FIGITEAKKLESKINKVF

SEQ ID NO: 59 - protein, 18 aa, Artificial Sequence
  Modified MTTE
FIGITELAKLESKINKVF

SEQ ID NO: 60 - protein, 18 aa, Artificial Sequence
  Modified MTTE
FIGITELKALESKINKVF

SEQ ID NO: 61 - protein, 18 aa, Artificial Sequence
  Modified MTTE
FIGITELKKAESKINKVF

SEQ ID NO: 62 - protein, 18 aa, Artificial Sequence
  Modified MTTE
FIGITELKKLASKINKVF

SEQ ID NO: 63 - protein, 18 aa, Artificial Sequence
  Modified MTTE
FIGITELKKLEAKINKVF

SEQ ID NO: 64 - protein, 18 aa, Artificial Sequence
  Modified MTTE
FIGITELKKLESAINKVF

SEQ ID NO: 65 - protein, 18 aa, Artificial Sequence
  Modified MTTE
FIGITELKKLESKANKVF

SEQ ID NO: 66 - protein, 18 aa, Artificial Sequence
  Modified MTTE
FIGITELKKLESKIAKVF

SEQ ID NO: 67 - protein, 18 aa, Artificial Sequence
  Modified MTTE
FIGITELKKLESKINAVF

SEQ ID NO: 68 - protein, 18 aa, Artificial Sequence
  Modified MTTE
FIGITELKKLESKINKAF

SEQ ID NO: 69 - protein, 18 aa, Artificial Sequence
  Modified MTTE
FIGITELKKLESKINKVA

SEQ ID NO: 70 - protein, 18 aa, Artificial Sequence
  Modified MTTE
YIGITELKKLESKINKVF

SEQ ID NO: 71 - protein, 18 aa, Artificial Sequence
  Modified MTTE
FLGITELKKLESKINKVF

SEQ ID NO: 72 - protein, 18 aa, Artificial Sequence
  Modified MTTE
FISITELKKLESKINKVF

SEQ ID NO: 73 - protein, 18 aa, Artificial Sequence
  Modified MTTE
FIGLTELKKLESKINKVF

SEQ ID NO: 74 - protein, 18 aa, Artificial Sequence
  Modified MTTE
FIGISELKKLESKINKVF

SEQ ID NO: 75 - protein, 18 aa, Artificial Sequence
  Modified MTTE
FIGITDLKKLESKINKVF

SEQ ID NO: 76 - protein, 18 aa, Artificial Sequence
  Modified MTTE
FIGITEIKKLESKINKVF

SEQ ID NO: 77 - protein, 18 aa, Artificial Sequence
  Modified MTTE
FIGITELRKLESKINKVF

SEQ ID NO: 78 - protein, 18 aa, Artificial Sequence
  Modified MTTE
FIGITELKRLESKINKVF

SEQ ID NO: 79 - protein, 18 aa, Artificial Sequence
  Modified MTTE
FIGITELKKIESKINKVF

SEQ ID NO: 80 - protein, 18 aa, Artificial Sequence
  Modified MTTE
FIGITELKKLETKINKVF

SEQ ID NO: 81 - protein, 18 aa, Artificial Sequence
  Modified MTTE
FIGITELKKLESRINKVF

SEQ ID NO: 82 - protein, 18 aa, Artificial Sequence
  Modified MTTE
FIGITELKKLESKLNKVF

SEQ ID NO: 83 - protein, 18 aa, Artificial Sequence
  Modified MTTE
FIGITELKKLESKIQKVF

SEQ ID NO: 84 - protein, 18 aa, Artificial Sequence
  Modified MTTE
FIGITELKKLESKINRVF

SEQ ID NO: 85 - protein, 18 aa, Artificial Sequence
  Modified MTTE
FIGITELKKLESKINKLF

SEQ ID NO: 86 - protein, 18 aa, Artificial Sequence
  Modified MTTE
FIGITELKKLESKINKVY

SEQ ID NO: 87 - protein, 19 aa, Gallus gallus
LEQLESIINFEKLAAAAAK

SEQ ID NO: 88 - protein, 20 aa, Artificial Sequence
  Scrambled SLP
DGLQGLLLGLRQRIETLEGK

SEQ ID NO: 89 - protein, 8 aa, Gallus gallus
SIINFEKL

SEQ ID NO: 90 - protein, 9 aa, Homo sapiens
ILLWQPIPV

SEQ ID NO: 91 - protein, 9 aa, Homo sapiens
YLPFRNCPR

SEQ ID NO: 92 - protein, 9 aa, Homo sapiens
LYCESVHNF

SEQ ID NO: 93 - protein, 9 aa, Homo sapiens
MMNDQLMFL

SEQ ID NO: 94 - protein, 9 aa, Homo sapiens
VLAGGFFLL

SEQ ID NO: 95 - protein, 10 aa, Homo sapiens
LLAVTSIPSV

SEQ ID NO: 96 - protein, 15 aa, Homo sapiens
TEDTMTKLRELSELS

SEQ ID NO: 97 - protein, 10 aa, Homo sapiens
YTLRVDCTPL

SEQ ID NO: 98 - protein, 18 aa, Artificial Sequence
  Scrambled MTTE
EKLINKLSKIFKGTIEVF

SEQ ID NO: 99 - protein, 10 aa, Artificial Sequence
  Spacer Sequence
AEKYARVRAK

SEQ ID NO: 100 - protein, 29 aa, Artificial Sequence
  B Cell Epitope Containing Peptide
FIGITELKKLESKINKVFAEKYARVRAKC

SEQ ID NO: 101 - protein, 15 aa, Homo sapiens
LLEFYLAMPFATPME

SEQ ID NO: 102 - protein, 11 aa, Artificial Sequence
  Spacer Sequence
AAKYARVRAKC

SEQ ID NO: 103 - protein, 28 aa, Artificial Sequence
  Scrambled MTTE with Spacer
EKLINKLSKIFKGTIEVFSSAFADVEAA

SEQ ID NO: 104 - protein, 28 aa, Artificial Sequence
  MTTE with Spacer
FIGITELKKLESKINKVFSSAFADVEAA

SEQ ID NO: 105 - protein, 27 aa, Artificial Sequence
  MTTE with Spacer
FIGITELKKLESKINKVFAAKYARVRA

SEQ ID NO: 106 - protein, 30 aa, Artificial Sequence
  Conjugate I SLP without TAP Sequence
NYARTEDFFQQQPPPGQDLFGIWSKVYDPL

SEQ ID NO: 107 - protein, 27 aa, Artificial Sequence
  Conjugate II SLP without TAP Sequence
LLHETDSAVAAARQIYVAAFTVQAAAE

SEQ ID NO: 108 - protein, 27 aa, Artificial Sequence
  Conjugate III SLP without TAP Sequence
SLSLGFLFLAAAGKVFRGNKVKNAQLA

SEQ ID NO: 109 - protein, 24 aa, Artificial Sequence
  Conjugate IV SLP without TAP Sequence
GMPEGDLVYTGNFSTQKVKMHIHS

SEQ ID NO: 110 - protein, 24 aa, Artificial Sequence
  Conjugate V SLP without TAP Sequence
KVFRGNKVKNYTLRVDCTPLMYSL